(19) United States
(12) Patent Application Publication
(10) Pub. No.: US 2022/0024978 A1
(43) Pub. Date: Jan. 27, 2022

(54) PEPTIDES COMPRISING OPIOID RECEPTOR AGONIST AND NK1 RECEPTOR ANTAGONIST ACTIVITIES

(71) Applicant: ARIZONA BOARD OF REGENTS ON BEHALF OF THE UNIVERSITY OF ARIZONA, Tucson, AZ (US)

(72) Inventors: Victor J. Hruby, Tucson, AZ (US); Aswini K. Giri, Tucson, AZ (US)

(73) Assignee: ARIZONA BOARD OF REGENTS ON BEHALF OF THE UNIVERSITY OF ARIZONA, Tucson, AZ (US)

(21) Appl. No.: 17/499,464

(22) Filed: Oct. 12, 2021

Related U.S. Application Data
(63) Continuation-in-part of application No. 15/121,270, filed on Aug. 24, 2016, now Pat. No. 11,141,452.
(60) Provisional application No. 61/943,820, filed on Feb. 24, 2014.

(51) Int. Cl. C07K 7/06

(52) U.S. Cl. CPC C07K 7/06; A61K 38/00

(57) ABSTRACT

The present invention relates generally to a compound having both agonist activity at opioid receptor(s) and antagonist activity at NK1 receptor, and methods for producing and using the same. This combination of activities provides several synergistic and/or beneficial effects such as enhanced potency in analgesic effect and reduction or inhibition of tolerance.

### CROSS-REFERENCE TO RELATED APPLICATIONS

This application is a continuation-in-part of U.S. patent application Ser. No. 15/121,270, filed Aug. 24, 2016, now U.S. Pat. No. 11,141,452, issued Oct. 12, 2021, which is a U.S. National Stage Patent Application of PCT Patent Application No. PCT/US15/17324, filed Feb. 24, 2015, which claims the priority benefit of U.S. Provisional Patent Application No. 61/943,820, filed Feb. 24, 2014, all of which are incorporated herein by reference in their entirety.

### STATEMENT REGARDING FEDERALLY FUNDED RESEARCH

This invention was made with government support under Grant Nos. P01 DA006284 and R01 DA013449 awarded by the National Institutes of Health. The government has certain rights in the invention.

### FIELD OF THE INVENTION

The present invention relates generally to a compound having both agonist activity at opioid receptor(s) and antagonist activity at NK1 receptor, and methods for producing and using the same. This combination of activities provides several synergistic and/or beneficial effects such as enhanced potency in analgesic effect and reduction or inhibition of tolerance.

### BACKGROUND OF THE INVENTION

Pain is caused by a highly complex perception of an aversive or unpleasant sensation, and the management of pain, mainly sustained and neuropathic pain, is a major challenge as millions of people all over the world suffer from such kind of pain every day. Opioids continue to be the backbone for the treatment of these pain states. However, constant opioid treatment is accompanied with serious undesirable effects including drowsiness and mental clouding, nausea and emesis, constipation and in many cases dependence and addiction. Continuous use of opioid therapy also develops analgesic tolerance and hyperalgesia in many patients. These unwanted effects significantly diminish the patients' quality of life. The mechanisms for these side effects are still largely unclear. Sustained pain states lead to neuroplastic modifications in both ascending and descending pathways in the spinal column in which there is both an augmented release of neurotransmitters (e.g., substance P) that intensify pain and increased expression of the corresponding receptors responsible for releasing those pain-promoting ligands. Currently used drugs for the management of prolonged and neuropathic pain mostly can only control pain and cannot neutralize against these induced neuroplastic modifications. Thus, it is found that the drugs currently in use as analgesic cannot work well in these pathological conditions.

Opioid drugs also are widely used following major surgery and to control pain of terminal diseases such as cancer, but its use is limited by several undesired side effects including nausea, vomiting, constipation, dizziness, system changes (neuroplasticity) due to prolonged pain or treatment by the opioid drugs and the development of tolerance and physical dependence, which mainly come through theμ opioid receptor. Because of these limitations the search for the novel type of analgesics which have strong pain controlling effect without development of tolerance and/or physical dependence has been performed for decades.

Opiates work in the brain at specific “opiate receptors.” Several types of the opiate receptors are known, but the main receptor for pain is called the μ receptor. Administering receptor agonists can cause full or partial stimulation or effect at the receptor, while administering antagonists blocks the effect of the receptor. It is widely accepted that a μ receptor agonist such as morphine has higher antinociceptive activity accompanied with high abuse liability. On the other hand, the activation of the δ opioid receptor has lower analgesic efficacy, but has reduced addictive potential. It is also generally known that the selective agonists at the δ opioid receptor have analgesic activity in numerous animal models with fewer adverse effects, though their efficacy is less potent than that of their widely-used μ counterparts. Thus, selective δ opioid agonists with enhanced analgesic activity are expected as a potent drug candidate for severe pain control.

Substance P is the preferred ligand for the neurokinin 1 (NK1) receptor and is known to contribute to chronic inflammatory pain and participate in central sensitization and associated hyperalgesia. In the pain states, substance P, which is an 11-amino acid polypeptide, is known as a major neurotransmitter of pain signals as well as the signals induced by opioid stimulation. Substance P and NK1 receptor expression increases after sustained opioid administration. Also, repeated morphine exposure results in enhanced levels of substance P in pain pathways both in vitro and in vivo, which could induce increased pain; increased pain could require increased pain-relief and thus be manifested as “antinociceptive tolerance”. Interestingly, co-administration of δ/μ opioid agonists and a substance P antagonist showed enhanced antinociceptive effect in acute pain states, and in prevention of opioid-induced tolerance in chronic trials. These results suggest that the signals through opioid receptors and neurokinin 1 (NK1) receptors are not independent, but have strong and critical interaction. Moreover, the mice lacking NK1 receptors, the preferred receptor of substance P, didn't show rewarding properties for opiates.

According to these observations, the use of multimodal combination analgesic therapies or therapies with a single molecule possessing the ability to interact with multiple analgesic targets has become attractive. Advantages of hybrid compounds system are developing bioactive compounds designed with a broad spectrum of receptor affinities and single administration of a chimeric compound instead of a specific ratio of two different compounds.

While opioid-based compounds are useful in treatment of pain, constant opioid treatment often is accompanied with serious undesirable effects including drowsiness and mental clouding, nausea and emesis, and constipation. Continuous use of opioid therapy also develops analgesic tolerance and hyperalgesia in many patients. These unwanted effects significantly diminish the patients' quality of life. The mechanisms for these side effects are still largely unclear. Sustained pain states lead to neuroplastic modifications in both ascending and descending pathways in the spinal column in which there is both an augmented release of neurotransmitters (e.g., substance P) that intensify pain and increased expression of the corresponding receptors responsible for releasing those pain-promoting ligands. Currently used drugs for the management of prolonged and neuropathic pain mostly can only control pain and cannot neutralize against these induced neuroplastic modifications. Thus, drugs currently in use as analgesic cannot work well in these pathological conditions.

Drug combinations have restrictions as therapeutics because of poor patient compliance, difficulties in drug metabolism, distribution, and possible drug-drug interactions. Therefore, there is a need for a compound that has both an opioid receptor agonist activity and neurokinin 1 (“NK1”) receptor antagonist activity in a single molecule. Such a compound would expect to provide advantages of activating opioid receptors, e.g., for treatment of acute and chronic neuropathic pain, while preventing undesired side-effects caused by activation of NK1 receptors, such as analgesic tolerance and hyperalgesia.

### SUMMARY OF THE INVENTION

Some aspects of the invention are based on the discovery by the present inventors that a compound (e.g., an oligopeptide) having both agonist activity at opioid receptor(s) (e.g., Mu-type receptor (MOR) and/or Delta-type receptor (DOR)), and antagonist activity at NK1 receptor is beneficiary over targeting a single receptor or using two different compounds to target opioid receptor(s) and NK1 receptor. This combination of activities addresses several fundamental biological effects such as enhanced potency in analgesic effect (e.g., for treatment of pain) and inhibition of opioid-induced tolerance. In particular, the present inventors have discovered that the combination of opioid receptor agonist and NK1 receptor antagonist activity in a single compound or molecule have synergistic effects in the management of prolonged pain states particularly pain states that involve higher substance P activity. Unless the context requires otherwise, the terms “compound,” “ligand,” and “oligopeptide” are used interchangeably herein and refers to molecules disclosed herein.

One particular aspect of the invention provides an oligopeptide of the formula:

AA¹-Q-Pro-AA²-AA³

where
AA¹ is Tyr or Dmt;
AA² is Leu or methylate Leu;
AA³ is Trp or methylated Trp each of which is covalently linked to optionally substituted benzyl amine or (optionally substituted phenyl)ethan-1-amine; and
Q is a moiety of the formula:

-(D)-NRAla-Phe′-NRGly-Tyr′-Pro-Ser-, or

-Pro-[Z]_{b}-Phe′-Pro-;

-AA⁴-AA⁵-AA⁶-AA⁷-Pro-Ser- (i)
AA⁴ is (D)-Ala or methylated (D)-Ala;
AA⁵ is Phe, Phe(4-F), methylated Phe, or methylated Phe(4-F);
AA⁶ is Gly or methylated Gly; and
AA⁷ is Tyr or Dmt; or

-Pro-(AA⁸)ₐ-AA⁹-Pro- (ii)
a is 0 or 1;
AA⁸ is Phe or Trp; and
AA⁹ is Phe, Phe(4-F), methylated Phe, or methylated Phe(4-F), provided at least one of AA², AA³, AA⁵, or AA⁹ is methylated.

In some embodiments, AA³ is covalently linked to benzyl amine, 3,5-di(trifluoromethyl)benzylamine, 1-phenylethan-1-amine, or 1-(3,5-di(trifluoromethyl)phenyl)ethan-1-amine. In one particular instance, AA³ is covalently linked to 3,5-di(trifluoromethyl)benzylamine or 1-(3,5-di(trifluoromethyl)phenyl)ethan-1-amine.

Still in other embodiments, Q is a moiety of the formula:

-AA⁴-AA⁵-AA⁶-AA⁷-Pro-Ser-

where AA⁴, AA⁵, AA⁶, and AA⁷ are as defined herein. As used herein, the terms “those defined above,” “those defined herein,” “as defined herein,” and “as defined above” are used interchangeably and when referring to a variable incorporates by reference the broad definition of the variable as well as any narrower definition(s), if any.

Yet in other embodiments, Q is a moiety of the formula:

-Pro-(AA⁸)ₐ-AA⁹-Pro-

wherein a, AA⁸, and AA⁹ are as defined herein.

Another aspect of the invention provides a method for treating pain comprising administering a subject in need of such a treatment a therapeutically effective amount of a compound of an oligopeptide of the formula:

-AA¹-Q-Pro-AA²-AA³

wherein
AA¹ is Tyr or Dmt;
AA² is Leu or methylate Leu;
AA³ is Trp or methylated Trp each of which is covalently linked to optionally substituted benzyl amine or (optionally substituted phenyl)ethan-1-amine; and
Q is a moiety of the formula:

-(D)-NRAla-Phe′-NRGly-Tyr′-Pro-Ser-, or

-Pro-[Z]_{b}Phe′-Pro-;

-AA⁴-AA⁵-AA⁶-AA⁷-Pro-Ser- (i)
AA⁴ is (D)-Ala or methylated (D)-Ala;
AA⁵ is Phe, Phe(4-F), methylated Phe, or methylated Phe(4-F);
AA⁶ is Gly or methylated Gly; and
AA⁷ is Tyr or Dmt; or

-Pro-(AA⁸)ₐ-AA⁹-Pro- (ii)
a is 0 or 1;
AA⁸ is Phe or Trp; and
AA⁹ is Phe, Phe(4-F), methylated Phe, or methylated Phe(4-F), provided at least one of AA², AA³, AA⁵, or AA⁹ is methylated.

In some embodiments, pain is an acute pain. Yet in other embodiments, pain is a chronic pain. Still in other embodiments, the method includes administering any one or more of the oligopeptides disclosed herein or a composition comprising the same.

Still other aspects of the invention provide a pharmaceutical composition comprising a pharmaceutically acceptable excipient and an oligopeptide of the formula:

AA¹-Q-Pro-AA²-AA³

wherein
AA¹ is Tyr or Dmt;
AA² is Leu or methylate Leu;
AA³ is Trp or methylated Trp each of which is covalently linked to optionally substituted benzyl amine or (optionally substituted phenyl)ethan-1-amine; and
Q is a moiety of the formula:

-(D)-NRAla-Phe′-NRGly-Tyr′-Pro-Ser-, or

-Pro-[Z]_{b}Phe′-Pro-;

-AA⁴-AA⁵-AA⁶-AA⁷-Pro-Ser- (i)
AA⁴ is (D)-Ala or methylated (D)-Ala;
AA⁵ is Phe, Phe(4-F), methylated Phe, or methylated Phe(4-F);
AA⁶ is Gly or methylated Gly; and
AA⁷ is Tyr or Dmt; or

-Pro-(AA⁸)ₐ-AA⁹-Pro- (ii)
a is 0 or 1;
AA⁸ is Phe or Trp; and
AA⁹ is Phe, Phe(4-F), methylated Phe, or methylated Phe(4-F), provided at least one of AA², AA³, AA⁵, or AA⁹ is methylated.

Still in other embodiments, the composition can include any or more of the oligopeptides disclosed herein.

### BRIEF DESCRIPTION OF THE DRAWINGS

FIG. 1 is a graph showing the results of paw withdrawal latency test after i.t. administration of oligopeptide AKG115.

FIG. 2 is a graph showing results of a tail flick latency after administration of oligopeptide AKG127 in accordance with the present invention.

FIG. 3 shows the percentage of antinociception at the same dose for oligopeptides AKG115 and AKG127.

### DETAILED DESCRIPTION OF THE INVENTION

According to the International Association for the Study of Pain (IASP), pain is defined as “an unpleasant sensory and emotional experience associated with actual or potential tissue damage or described in terms of such damage”. Pain can be classified in numerous ways and accordingly, different types of pain are discussed in the literature. Pain has significant physical, economic and social impact. Approximately 1.5 billion people around the globe suffer from chronic pain. The costs associated with pain treatment are much higher than that involved for the treatment of heart disease or cancer. Generally, acute pain associated with accidental injury or surgery is cured. But nearly 50% of patients who have gone through surgery face chronic pain. Under-treatment of postsurgical acute pain has been found as a major reason for moderate to severe or even extreme pain in two thirds of these patients.

In spite of having many serious side effects including respiratory depression, sedation, constipation, physical dependence and development of tolerance, opioid agonists have long been the mainstay analgesics for the treatment of various pain states because of their potency, efficacy and availability. Three classical opioid receptors, namely μ-, δ- and κ-opioid receptors (MOR, DOR and KOR, respectively), have been identified in the central nervous system by pharmacological studies. The common opioid drugs including morphine, codeine, oxycodone, methadone, heroin, morphine-6β-glucuronide (M6G), fentanyl, etc., which are used clinically for analgesic effects, mainly targets the MOR. Most studies have confirmed that the μ-opioid receptor is primarily responsible for the antinociceptive activity. However, a number of studies have suggested that ligands with dual μ- and δ-agonist activities display better biological profiles compared to the ones acting selectively on MOR. There is also evidence that the presence of DOR agonists can improve the analgesic efficacy of MOR agonists. KORs, broadly found in the spinal cord, the dorsal ganglia, the periphery and the supraspinal regions, are associated with pain modulation.

To overcome the difficulties in pain treatment described above, new approaches to drug design are needed to deal with recent observations that in the development of prolonged and neuropathic pain states, there are critically important changes in the expressed genome in ascending and descending pain pathways, and in the CNS that result from up regulation of neurotransmitter receptors and their ligands that are stimulatory and thus can cause pain. These anti-opioid ligands and receptors need to be considered in drug design. Therefore, there is a need to develop approaches to design ligands that are multivalent and therefore can act at two, three or more receptors all with a single molecule. The present invention provides such new approaches.

The main clinically used drugs for the treatment of pain are opioid agonists. Although most of the currently used opioid drugs can act upon all three subtypes of opioid receptors, the drugs' analgesic effects are mainly due to the activation of MOR present in the central nervous system (CNS). One of the key reasons of having limited a number of centrally acting drugs is due to the presence of the blood-brain barrier (BBB), which put forward some constraint for foreign molecules to enter into the brain. The BBB permits hydrophobic and selected molecules to pass through it. But hydrophobic agents are difficult to transport via blood which requires more hydrophilic nature of the drug candidates. These two opposite requirements by the blood and the BBB have made it a challenging job for scientists to discover and develop new drugs, which can be delivered into the CNS. Another issue associated with development of centrally acting opioid drugs is their metabolic stability. This is because of the fact that therapeutic agents should have half-lives in the acceptable range so that they can interact with their biological targets for a sufficient duration of time to produce the desired response.

Some aspects of the invention are based on a discovery by the present inventors of oligopeptides having two or three different receptor activities a single molecule or a compound. Such compounds also have appropriate metabolic and pharmacological properties to provide a synergistic effect on pain management. The ligand has potent analgesic affects not only in acute pain but also in prolonged and neuropathic pain states without the development of unwanted side effects. However, prior to the discovery by the present inventors, it was still largely unclear what binding ratio(s) for the receptors should be ideal to achieve the desired biological profile. To address these issues an innovative approach has been taken to design, synthesize and evaluate the detailed biological profile of the ligands showing different kind of ratios of binding affinity for all three receptors with appropriate functional activities. In particular, the present invention employs an innovative approach to design, synthesize and evaluate the detail biological profile of the ligands showing different kind of ratios of binding affinity for all three receptors with appropriate functional activities.

Some aspects of the invention are based on adjacent and overlapping pharmacophores, in which an opioid agonist pharmacophore is placed at the N-terminus and the NK1 antagonist pharmacophore sits at the C-terminus of a single peptide derived ligand. The opioid pharmacophore of these multivalent/multifunctional ligands were designed based on the sequences of well-known opioid agonist ligands including enkephalin (Met-enkephalin: H-Tyr-Gly-Gly-Phe-Met (SEQ ID NO:1) and Leu-Enkephalin: H-Tyr-Gly-Gly-Phe-Leu (SEQ ID NO:2)), DAMGO, (H-Tyr-D-Ala-Gly-N-MePhe-Gly-OH (SEQ ID NO:3)), dermorphin (H-Tyr-D-Ala-Phe-Gly-Tyr-Pro-Ser-NH₂ (SEQ ID NO:4)), morphiceptin (H-Tyr-Pro-Phe-Pro-NH₂ (SEQ ID NO:5)), and endomorphins (Endomorphin 1: H-Tyr-Pro-Trp-Phe-NH₂ (SEQ ID NO:6); Endomorphin 2: H-Tyr-Pro-Phe-Phe-NH₂ (SEQ ID NO:7)), while the NK1 antagonist pharmacophore (i.e. -Pro-Leu-Trp-NH-Bn(3′,5′-(CF₃)₂))) was adopted from the previously published pharmacophore (e.g., TY027: H-Tyr-D-Ala-Gly-Phe-Met-Pro-Leu-Trp-NH-Bn(3′,5′-(CF₃)₂) (SEQ ID NO:8)) (Hruby et al. U.S. Pat. No. 8,026,218) for the same kind of activity. The two pharmacophores are joined directly or by a linker, which might be working as an address region for both pharmacophores as well as a spacer between them. It should be highlighted that the designed multivalent/multifunctional ligands have additional rewards over a cocktail of individual drugs for easy administration, a simple ADME property and no drug-drug interactions. Local concentration is also expected to be higher than that in the coadministration of drug cocktails as the expression of the NK1 and opioid receptors as well as the neurotransmitters show a significant degree of overlap in the central nervous system, resulting to synergies in potency and efficacy. Previous studies have shown that the lead bifunctional compounds, TY005 (H-Tyr¹-D-Ala²-Gly³-Phe⁴-Met⁵-Pro⁶-Leu⁷-Trp⁸-O—NH-Bn(3′,5′-(CF₃)₂ (SEQ ID NO:9)) and TY027 (H-Tyr¹-D-Ala²Gly³-Phe⁴-Met⁵-Pro⁶-Leu⁷-Trp⁸-NH-Bn(3′,5′-(CF₃)₂ (SEQ ID NO:8)) are capable to treat neuropathic pain in a rodent model with blood brain barrier permeability, no development of opioid-induce tolerance, and no development of reward liability, supporting our hypothesis that a single ligand containing opioid agonist/NK1 antagonist activities is effective against neuropathic pain[18]. It should be noted here that the above-mentioned ligands have shown their binding affinity and functional activity on both DOR and MOR, but with some selectivity for the former one over the latter one while maintaining their biological profile at the NK1 receptor. Surprisingly, we have found that combining two activities, i.e., opioid agonists and NK1 antagonist, on one ligand provides enhanced metabolic and pharmacological properties including increased blood-brain barrier penetration not observed when an opioid agonist and an NK1 antagonist are administered separately.

More particularly, the present inventors have shown that agonist activities at Mu-type and Delta-type opioid receptors (MOR and DOR), and antagonist activity at NKI is beneficiary over targeting a single receptor. This combination explains several fundamental biological effects such as enhanced potency in acute pain models and inhibition of opioid-induced tolerance in chronic tests using rats. A study revealed that NK1 knockout mice did not show the rewarding properties of morphine. Thus, the combination of opioid receptor agonist and NK1 receptor antagonist activity may have synergistic effects in the management of prolonged pain states that involve higher substance P activity. Drug combinations have restrictions as therapeutics because of poor patient compliance, difficulties in drug metabolism, distribution, and possible drug-drug interactions.

Oligopeptides of the invention combine these two or three different activities in one ligand to provide appropriate metabolic and pharmacological properties. The oligopeptides of the invention have potent analgesic affects not only in acute pain but also in prolonged and neuropathic pain states without the development of (or with a significantly reduced level of incidences of) unwanted side effects.

In one aspect of the invention, there is provided a compound for treatment of pain comprising a single multivalent/multifunctional ligand with agonist activity at opioid receptors and with antagonist activity at NK1 receptors, joined by a linker, or by a covalent bond. In such aspect opioid pharmacophore moiety preferably is selected from the group consisting DAMGO, dermorphin, morphiceptin, and endomorphin, and/or the linker preferably has a length of one to three amino acids.

In one aspect of the invention, the opioid pharmacophore moiety is cyclic.

In another aspect of the invention, the compound has the structure:

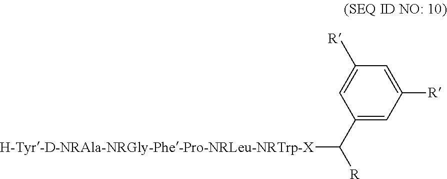

Tyr′=Tyr and its derivatives, e.g., Dmt etc.; Phe′=Phe and its derivatives, e.g., NMePhe, Phe(4-F) etc.; R=H, Me, etc.; R′=H, CH₃, CF₃ etc.; X=NH, NMe, etc., or an analog thereof selected from the group consisting of: H-Tyr-D-Ala-Gly-NMePhe-Pro-Leu-Trp-NH-Bn(3′,5′-(CF₃)₂) (SEQ ID NO:11); H-Dmt-D-Ala-Gly-NMePhe-Pro-Leu-Trp-NH-Bn(3′,5′-(CF₃)₂) (SEQ ID NO:12); H-Dmt-D-Ala-Gly-Phe-Pro-Leu-Trp-NH-Bn(3′,5′-(CF₃)₂) (SEQ ID NO:13); H-Dmt-D-Ala-Gly-Phe(4-F)-Pro-Leu-Trp-NH-Bn(3′,5′-(CF₃)₂) (SEQ ID NO:14); H-Dmt-D-Ala-Gly-NMePhe(4-F)-Pro-Leu-Trp-NH-Bn(3′,5′-(CF₃)₂) (SEQ ID NO:15); H-Dmt-D-Ala-Gly-Phe(4-Cl)-Pro-Leu-Trp-NH-Bn(3′,5′-(CF₃)₂) (SEQ ID NO:16); H-Dmt-D-Ala-Gly-Phe(4-Br)-Pro-Leu-Trp-NH-Bn(3′,5′-(CF₃)₂) (SEQ ID NO:17); and H-Dmt-D-Ala-Gly-Phe(4-I)-Pro-Leu-Trp-NH-Bn(3′,5′-(CF₃)₂) (SEQ ID NO:18).

In another aspect of the invention the compound has the structure:

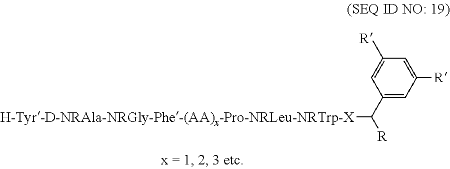

Tyr′=Tyr and its derivatives, e.g., Dmt etc.; Phe′=Phe and its derivatives, e.g., NMePhe, Phe(4-F) etc.; R=H, Me, etc.; AA=natural/unnatural amino acid e.g., Nle, Gly, β-Ala, γ-Abu, Ahx, 4-Amb, 4-Abz, 4-Apac, 4-Ampa etc.; R′=H, CH₃, CF₃ etc.; X=NH, NMe, etc.

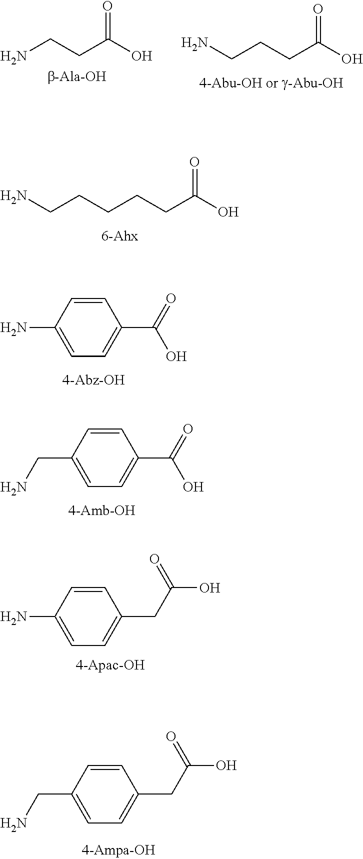

and an analog thereof selected from the group consisting of: H-Tyr-D-Ala-Gly-NMePhe-Nle-Pro-Leu-Trp-NH-Bn(3′,5′-(CF₃)₂) (SEQ ID NO:20); H-Tyr-D-Ala-Gly-NMePhe-Gly-Pro-Leu-Trp-NH-Bn(3′,5′-(CF₃)₂) (SEQ ID NO:21); H-Tyr-D-Ala-Gly-Phe(4-F)-Gly-Pro-Leu-Trp-NH-Bn(3′,5′-(CF₃)₂) (SEQ ID NO:22); H-Tyr-D-Ala-Gly-NMePhe(4-F)-Gly-Pro-Leu-Trp-NH-Bn(3′,5′-(CF₃)₂) (SEQ ID NO:23); H-Tyr-D-Ala-Gly-NMePhe-β-Ala-Pro-Leu-Trp-NH-Bn(3′,5′-(CF₃)₂) (SEQ ID NO:24); H-Tyr-D-Ala-Gly-NMePhe-γ-Abu-Pro-Leu-Trp-NH-Bn(3′,5′-(CF₃)₂) (SEQ ID NO:25); H-Tyr-D-Ala-Gly-NMePhe-[[4]]6-Ahx-Pro-Leu-Trp-NH-Bn(3′,5′-(CF₃)₂) (SEQ ID NO:26); H-Tyr-D-Ala-Gly-NMePhe-4-Amb-Pro-Leu-Trp-NH-Bn(3′,5′-(CF₃)₂) (SEQ ID NO:27); H-Tyr-D-Ala-Gly-NMePhe-4-Abz-Pro-Leu-Trp-NH-Bn(3′,5′-(CF₃)₂) (SEQ ID NO:28); H-Tyr-D-Ala-Gly-NMePhe-4-Apac-Pro-Leu-Trp-NH-Bn(3′,5′-(CF₃)₂) (SEQ ID NO:29); and H-Tyr-D-Ala-Gly-NMePhe-4-Ampa-Pro-Leu-Trp-NH-Bn(3′,5′-(CF₃)₂) (SEQ ID NO:30).

In still yet another aspect of the invention, the compound has the structure:

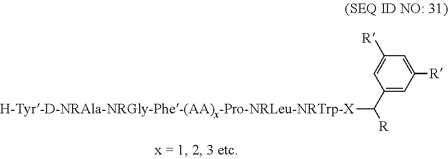

Tyr′=Tyr or its derivative, e.g., Dmt etc.; Phe′=Phe or its derivative, e.g., NMePhe, Phe(4-F), NMePhe(4-F), etc.; R=H, Me, etc.; AA=natural/unnatural amino acid e.g., Ser, D-Ser, Homo-Ser, Lys, Orn, Dab, Dap, Ser-4-Apac, Asn, D-Asn, Gln, D-Gln, Gln-4-Apac, etc.; R′=H, CH₃, CF₃ etc.; X=NH, NMe, etc., or an analog thereof selected from: SEQ ID NO:31, where Tyr′=Tyr, R=H, Phe′=Phe, x=1, AA=Ser; X=NH, R′=CF₃; Tyr′=Tyr, R=H, Phe′=Phe, x=1, AA=Homo-Ser; X=NH, R′=—CF₃; Tyr′=Tyr, R=H, Phe′=Phe, x=1, AA=Ser; X=NH, R′=CF₃; Tyr′=Tyr, R=H, Phe′=NMePhe, x=1, AA=Ser; X=NH, R′=—CF₃; Tyr′=Dmt, R=H, Phe′=NMePhe, x=1, AA=Ser; X=NH, R′=—CF₃; Tyr′=Tyr, R=H, Phe′=Phe(4-F), x=1, AA=Ser; X=NH, R′=—CF₃; Tyr′=Tyr, R=H, Phe′=NMePhe(4-F), x=1, AA=Ser; X=NH, R′=—CF₃; Tyr′=Dmt, R=H, Phe′=NMePhe(4-F), x=1, AA=Ser; X=NH, R′=—CF₃; Tyr′=Tyr, R=H, Phe′=NMePhe, x=2, (AA)x=Ser-Gly; X=NH, R′=—CF₃; Tyr′=Dmt, R=H, Phe′=NMePhe, x=2, (AA)x=Ser-Gly; X=NH, R′=—CF₃; Tyr′=Tyr, R=H, Phe′=Phe, x=1, AA=Asn; X=NH, R′=—CF₃; Tyr′=Tyr, R=H, Phe′=Phe, x=1, AA=D-Asn; X=NH, R′=—CF₃; Tyr′=Tyr, R=H, Phe′=Phe, x=1, AA=Gln; X=NH, R′=—CF₃; Tyr′=Tyr, R=H, Phe′=Phe, x=1, AA=D-Gln; X=NH, R′=—CF₃; Tyr′=Tyr, R=H, Phe′=NMePhe, x=1, AA=Gln; X=NH, R′=—CF₃; Tyr′=Dmt, R=H, Phe′=NMePhe, x=1, AA=Gln; X=NH, R′=—CF₃; Tyr′=Tyr, R=H, Phe′=Phe(4-F), x=1, AA=Gln; X=NH, R′=—CF₃; Tyr′=Tyr, R=H, Phe′=NMePhe(4-F), x=1, AA=Gln; X=NH, R′=—CF₃; Tyr′=Dmt, R=H, Phe′=NMePhe(4-F), x=1, AA=Gln; X=NH, R′=CF₃; Tyr′=Tyr, R=H, Phe′=NMePhe, x=2, (AA)x=Gln-Gly; X=NH, R′=CF₃; Tyr′=Dmt, R=H, Phe′=NMePhe, x=2, (AA)x=Gln-Gly; X=NH, R′=—CF₃.

In still yet another aspect of the invention the compound has the structure:

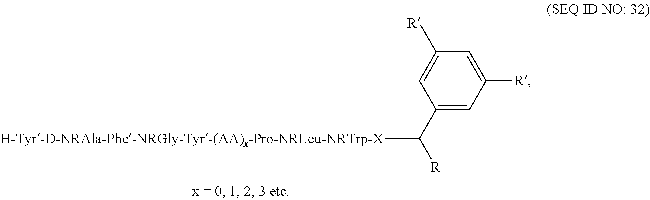

Tyr′=Tyr and its derivatives, e.g., Dmt etc.; Phe′=Phe and its derivatives, e.g., NMePhe, Phe(4-F), etc.; R=H, Me, etc.; AA=natural/unnatural amino acid, e.g., 4-Amb, 4-Apac, Lys, etc.; X=NH, NMe, etc.; R′=H, CH₃, CF₃ etc.

Exemplary compounds of SEQ ID NO:32 include compounds where (i) R=H, Phe′=Phe, Tyr′=Tyr, x=0, X=NH, R′=CF₃; (ii) R=H, Phe′=Phe, Tyr′=Tyr, x=0, X=NMe, R′=CF₃; (iii) R=H, Phe′=Phe, first Tyr′=Dmt, second Tyr′=Tyr, x=0, X=NH, R′=CF₃; (iv) R=H, Phe′=Phe(p-F), Tyr′=Tyr, x=0, X=NH, R′=CF₃; and (v) R=H, Phe′=Phe(p-F), first Tyr′=Dmr, second Tyr′=Tyr, x=0, X=NH, R′=CF₃.

In a further aspect of the invention the compound has the structure:

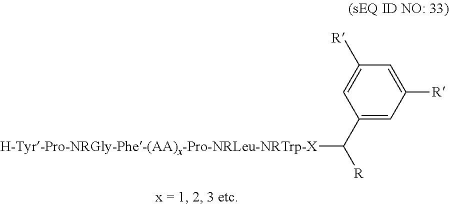

where Tyr′=Tyr or its derivative e.g., Dmt etc.; Phe′=Phe or its derivative, e.g., NMePhe, Phe(4-F) etc.; R=H, Me etc.; AA=natural/unnatural amino acid e.g., AA=4-Amb, 4-Apac, Lys, etc.; X=Nh, Nme etc., or an analog thereof selected from H-Tyr-Pro-Phe-Pro-Leu-Trp-NH-Bn(3′,5′-(CF₃)₂) (SEQ ID NO:34); H-Tyr-Pro-Phe-Pro-Pro-Leu-Trp-NH-Bn(3′,5′-(CF₃)₂) (SEQ ID NO:35); H-Tyr-Pro-Phe-Gly-Nle-Pro-Leu-Trp-NH-Bn(3′,5′-(CF₃)₂) (SEQ ID NO:36); H-Tyr-Pro-Phe-Pro-4-Amb-Pro-Leu-Trp-NH-Bn(3′,5′-(CF₃)₂) (SEQ ID NO:37); H-Tyr-Pro-Phe-Pro-4-Ampa-Pro-Leu-Trp-NH-Bn(3′,5′-(CF₃)₂) (SEQ ID NO:38); H-Tyr-Pro-Phe-Gly-Pro-Leu-Trp-NH-Bn(3′,5′-(CF₃)₂) (SEQ ID NO:39); H-Tyr-Pro-Phe-NMeGly-Pro-Leu-Trp-NH-Bn(3′,5′-(CF₃)₂) (SEQ ID NO:40); H-Tyr-Pro-Gly-Phe-Pro-Leu-Trp-NH-Bn(3′,5′-(CF₃)₂) (SEQ INO:41); H-Tyr-Pro-Gly-NMePhe-Pro-Leu-Trp-NH-Bn(3′,5′-(CF₃)₂) (SEQ ID NO:42).

In a further aspect of the invention the compound has the structure:

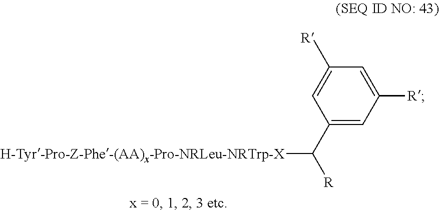

where Tyr′=Tyr or its derivative, e.g., Dmt etc.; Z=Phe′ or Trp′ or absent; Trp′=Trp or its derivative, e.g., NMeTrp etc.; Phe′=Phe or its derivative, e.g., NMePhe, Phe(4-F) etc.; R=H, Me etc.; AA=natural/unnatural amino acid., e.g., AA=4-Amb, 4-Apac, Lys, etc.; X=NH, NMe etc., or an analog thereof selected from: H-Tyr-Pro-Trp-Phe-Pro-Leu-Trp-NH-Bn(3′,5′-(CF₃)₂) NO:44); H-Tyr-Pro-Phe-Phe-Pro-Leu-Trp-NH-Bn(3′,5′-(CF₃)₂) (SEQ ID NO:45); H-Tyr-Pro-Trp-NMePhe-Pro-Leu-Trp-NH-Bn(3′,5′-(CF₃)₂) (SEQ ID NO:46); H-Tyr-Pro-Phe-NMePhe-Pro-Leu-Trp-NH-Bn(3′,5′-(CF₃)₂) (SEQ ID NO:47); H-Tyr-Pro-NMeTrp-NMePhe-Pro-Leu-Trp-NH-Bn(3′,5′-(CF₃)₂) (SEQ ID NO:48); H-Tyr-Pro-NMePhe-NMePhe-Pro-Leu-Trp-NH-Bn(3′,5′-(CF₃)₂) (SEQ ID NO:49); H-Tyr-D-Ala-Gly-Phe-Pro-Leu-Trp-NMe-Bn(3′,5′-(CF₃)₂) (SEQ INO:50); H-Tyr-D-Ala-Phe-Pro-Leu-Trp-NMe-Bn(3′,5′-(CF₃)₂) (SEQ ID NO:51).

The invention also provides a pharmaceutical composition comprising the compound as above described in a pharmaceutical-acceptable carrier.

The invention also provides a method for treating pain which comprises administering an effective amount of the above-described composition to an individual in need of treatment, as needed, preferably in a dose range of 1 mg/Kg to 100 mg/Kg.

The invention also provides a method for forming compound as above described, comprising the steps of solid phase peptide synthesis, cyclization via coupling of appropriate functional groups on solid phase, C-terminal modification and removal of all protecting group in solution phase.

The compounds of the present invention, salts, and derivatives thereof can be incorporated into pharmaceutical compositions suitable for administration. Such compositions typically comprise the compound and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Modifications can be made to the compound of the present invention to affect solubility or clearance of the compound. These molecules may also be synthesized with D-amino acids to increase resistance to enzymatic degradation. If necessary, the compounds can be co-administered with a solubilizing agent, such as cyclodextran.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Synthesis and characterization of ligands: All linear peptides were synthesized on solid phase using 2-chlorotrityl chloride resin (loading: 1.02 mmol/g) via Fmoc/tBu approach. All steps during solid phase synthesis were performed in frited syringes. N-methylation on desired amino acid was performed on solid phase. C-terminal amidation was conducted in solution phase.

Loading of the first amino acid on the resin: Chlorotrityl resin (0.102 mmol) was swelled in dry dichloromethane (DCM) for 1 hour at room temperature. After swelling, dry DCM was expelled from the syringe and the resin was washed with DCM (1 mL, 3×1 min). It was then ready for the first amino acid coupling. Pre-generated (by treating with 5.0 equiv. DIPEA) carboxylate of Fmoc-Trp(Boc)-OH (1.2 equiv.) in dry DCM (1.0 mL) was loaded onto the resin by substituting chloride from the resin. After the coupling of first amino acid, methanol (0.1 mL) was added to the mixture and was shaken for 15 minutes in order to cap any unreacted chloride present in the resin. It was then washed with DCM (1 mL, 5×1 min) and DMF (1 mL, 4×1 min).

Deprotection: Following the washes, deprotection of Fmoc group was performed. This was done by stirring the resin with 20% piperidine in DMF for 8 minutes, followed by 12 minutes. A DMF wash (1 mL, 1 min) was performed in between the two deprotection steps to remove side products. After the second piperidine treatment, resin washes were performed with DMF (1 mL, 3×1 min), DCM (1 mL, 3×1 min), and DMF (1 mL, 3×1 min) before the next coupling. These steps were repeated after coupling of each Fmoc protected amino acid in the peptide sequence.

Coupling: For the coupling of the remaining amino acids, HCTU (3.0 equiv. and in case of primary amine) or HATU/HOAt (3.0 equiv. of each, in case of secondary amine) was used as coupling reagents and DIPEA (6.0 equiv.) as base. All couplings involving primary amines were carried out in DMF while coupling of secondary amine was performed in NMP. Between each coupling, resin washes were performed with DMF (1 mL, 3×1 min), DCM (1 mL, 3×1 min), and DMF (1 mL, 3×1 min).

After each coupling or deprotection, the Kaiser/chloranil test was performed to determine whether or not amino acid coupling or Fmoc deprotection was successful. Kaiser tests were run for primary amino acids and chloranil tests for secondary amino acids (e.g. proline and methylated amino acids). A negative test after each coupling suggests that the reaction was complete. After deprotection, the same test should be positive.

N-Methylation of amino acids: After Fmoc deprotection of the desired amino acid that will be N-methylated, o-NBS protection, N-methylation, and then o-NBS deprotection were performed.

o-NBS protection: After Fmoc deprotection, the resin was washed with DMF, DCM, then NMP (3×1 min each). NMP was drained out from the syringe. NMP (1 mL) was added to the resin followed by the addition of o-NBS-Cl (4 equiv.) and sym-collidine (10.0 equiv.). It was stirred for 15 minutes. The same step was repeated for one more time after filtering and washing the resin with NMP (1 mL, 1×1 min) in between. It was then washed with NMP (1 mL, 5×1 min) and then used for N-methylation.

N-methylation (DBU mediated method): DBU (1,8-diazabicyclo(5,4,0)undec-7-ene) (3.0 equiv.) in NMP (1 mL) was treated with the resin for 3 minutes. Afterwards and without filtering, DMS (10.0 equiv.) was added directly to the syringe containing resin and DBU solution and stir for another 3 min. The resin was then filtered and washed with NMP (1×1 min). This step was repeated once followed by filtration, and washing with NMP (5×1 min). The resultant resin bound peptide with N-methylation on amino acid was used for o-NBS deprotection.

o-NBS deprotection: NMP (1 mL), 2-mercaptoethanol (10.0 equiv.), and DBU (5.0 equiv.) were added to the syringe and the resin was treated for 5 min. The resin was filtered and washed with NMP (1 mL, 1×1 min). The procedure was repeated one more time and then the resin was filtered and washed with NMP (5×1 min).

General procedure for carbon-carbon double bond formation: After completing the linear sequence of the peptide, the resin-bound peptide was dried under vacuum, transferred to a 3-neck round bottom flask and suspended in approximately dry dichloromethane (10 mL/0.1 mmol of resin bound peptide). The mixture was kept under an argon atmosphere, and argon gas was bubbled into the reaction mixture for 30 minutes. Grubbs Catalyst 2nd Generation i.e. Dichloro[1,3-Bis(2,4,6-trimethylphenyl)-2-imidazolidinylidene](3-methyl-2-butenylidene)(tricyclohexylphosphine)ruthenium(II) (20 mol % with respect to the resin-bound peptide) was added to the reaction mixture and argon was again bubbled through the solution for an additional 30 minutes. The reaction mixture was then refluxed for 48 h. DMSO (50 equivalents with respect to the catalyst) was then added to the reaction mixture after allowing it to cool to room temperature. The reaction mixture was then stirred for an additional 24 hours. The resin-bound peptide was filtered and washed with DMSO, dichloromethane, and MeOH (3×5). The resin-bound peptide was dried under vacuum and used for next step. This method was used for making cabocycle-, lactone-, carbamate-based cyclic ligands.

Cleaving peptide from the resin: DIPEA (0.200 mL) was added to a centrifuge tube to trap excess TFA while collecting the peptide. The resin was stirred on a shaker with 1% TFA (2 mL/0.102 mmol of starting resin) in DCM (3×5 min) on the shaker. The resin was rinsed in between cleavage with small amounts of DCM. The peptide containing solution was collected in the centrifuge tube. Resin became darker with each TFA treatment. Volatiles were evaporated from the centrifuge tube by flushing the resulting solution with argon.

Amidation: The crude peptide was dissolved in dry DMF (1 mL) followed by addition of HATU (1.0 equiv.), HOAt (1.0 equiv.), DIPEA (4.0 equiv), and 3,5-bis(trifluoromethyl)benzylamine (1.1 equiv.), respectively and mixture was stirred for overnight. Workup: KHSO₄ (0.5 M in H₂O, 5 mL) was added to reaction mixture followed by extraction with DCM (3×15 mL). The combined organic extract was taken into a separatory funnel and was washed with brine (1×15 mL). The organic part was washed with NaHCO₃ (1×15 mL) followed by another brine wash. The final organic solution was dried over anhydrous sodium sulfate; gravity filtrated, and then evaporated under pressure to remove DCM in a round bottom flask (RBF).

Removal of Boc/^{t}Bu protecting groups: The crude peptide was treated for 1 h with a cleavage cocktail containing 82.5% TFA, 5% H₂O, thioanisol, 5% phenol, and 2.5% 1,2-ethanedithiol to remove Boc/tBu protecting groups. After 1 h, the solution was flushed with argon to evaporate volatiles.

Precipitation: Hexanes wash (3×15 mL) was performed to remove low polar materials by vortexing the mixture with hexanes followed by centrifugation at 3300 rpm (3×5 min), each time replacing the hexanes layer. Washes with hexanes and dimethyl ether mixture (30:70, 3×15 mL) gave white precipitate in 80-100% as crude yield. Purification of crudes using RP-HPLC furnished the pure ligands in 20-40% overall yield for lilear peptides and 10-20% overall yield for cyclic peptides.

### Methods for In Vitro Study

hNK1/CHO Cell Membrane Preparation and Radioligand Binding Assay: Recombinant hNK1/CHO cells were grown to confluency in 37 ° C., 95% air and 5% CO₂, humidified atmosphere, in a Forma Scientific (Thermo Forma, OH) incubator in Ham's F12 medium supplemented with 10% fetal bovine serum, 100 U/mL penicillin, 100 m/mL streptomycin, and 500 m/mL geneticin. The confluent cell monolayers were then washed with Ca²⁺, Mg²⁺-deficient phosphate-buffered saline (PD buffer) and harvested in the same buffer containing 0.02% EDTA. After centrifugation at 2700 rpm for 12 min, the cells were homogenized in ice-cold 10 mM Tris-HCl and 1 mM EDTA, pH 7.4, buffer. A crude membrane fraction was collected by centrifugation at 18000 rpm for 12 min at 4° C., the pellet was suspended in 50 mM Tris-Mg buffer, and the protein concentration of the membrane preparation was determined by using Bradford assay.

Bradford assay: Six different concentrations of the test compound were each incubated, in duplicates, with 20 μg of membrane homogenate, and 0.5 nM [³H] SP (135 Ci/mmol, Perkin-Elmer, United States) in 1 mL final volume of assay buffer (50 mM Tris, pH 7.4, containing 5 mM MgCl₂, 50 μg/mL bacitracin, 30 μM bestatin, 10 μM captopril, and 100 μM phenylmethylsulfonylfluoride) SP at 10 μM was used to define the nonspecific binding. The samples were incubated in a shaking water bath at 25° C. for 20 min. The reaction was terminated by rapid filtration through Whatman grade GF/B filter paper (Gaithersburg, MD) presoaked in 1% polyethyleneimine, washed four times each with 2 mL of cold saline, and the filter bound radioactivity was determined by liquid scintillation counting (Beckman LS5000 TD).

Data Analysis: Analysis of data collected from three independent experiments performed in duplicates is done using GraphPad Prizm 4 software (GraphPad, San Diego, Calif.). Log IC₅₀ values for each test compound were determined from nonlinear regression. The inhibition constant (Ki) was calculated from the antilogarithmic IC₅₀ value by the Cheng and Prusoff equation.

Guinea Pig Isolated Ileum/Longitudinal Muscle with Myenteric Plexus (GPI/LMMP): Male Hartley guinea pigs under CO₂ anesthesia were sacrificed by decapitation and a non-terminal portion of the ileum removed. The longitudinal muscle with myenteric plexus (LMMP) was carefully separated from the circular muscle and cut into strips as described previously (Porreca and Burks, 1983). These tissues were tied to gold chains with suture silk and mounted between platinum wire electrodes in 20 mL organ baths at a tension of 1 g and bathed in oxygenated (95:5 O₂:CO₂) Kreb's bicarbonate buffer at 37° C. They were stimulated electrically (0.1 Hz, 0.4 msec duration) at supramaximal voltage. Following an equilibration period, compounds were added cumulatively to the bath in volumes of 14-60:l until maximum inhibition was reached. A dose-response curve of PL-017 was constructed to determine tissue integrity before analog testing.

Mouse Isolated Vas Deferens Preparation: Male ICR mice under CO2 anesthesia were sacrificed by cervical dislocation and the vasa differentia removed. Tissues were tied to gold chains with suture silk and mounted between platinum wire electrodes in 20 mL organ baths at a tension of 0.5 g and bathed in oxygenated (O₂:CO₂=95:5) magnesium free Kreb's buffer at 37° C. They were stimulated electrically (0.1 Hz, single pulses, 2.0 msec duration) at supramaximal voltage as previously described[44]. Following an equilibration period, compounds were added to the bath cumulatively in volumes of 14-60:l until maximum inhibition was reached. A dose-response curve of DPDPE was constructed to determine tissue integrity before analog testing.

Agonist and Antagonist Testing: Compounds were tested as agonists by adding cumulatively to the bath until a full dose-response curve was constructed or to a concentration of 1 M. Compounds were tested as antagonists by adding to the bath 2 minutes before beginning the cumulative agonist dose-response curves of the delta (DPDPE) or mu (PL-017) opioid agonists.

Analysis: Percentage inhibition was calculated using the average tissue contraction height for 1 min preceding the addition of the agonist divided by the contraction height 3 min after exposure to the dose of agonist. IC₅₀ values represent the mean of not less than 4 tissues. IC₅₀ and Eₘₐₓ estimates were determined by computerized nonlinear least-squares analysis (FlashCalc).

In vitro metabolic stability: A stock solution (50 mg/mL in DMSO) of each compound in study was made. It was diluted 1000-fold into rat plasma (Pel-Freez Biologicals, Rogers, Ak.) resulting in an incubation concentration of 50 μg/mL. Incubation temperature was 37° C. 200 μL of aliquots were pipetted out at different time points (i.e. 1 min, 10 min, 30 min, 1 h, 2 h, 4 h, 6 h, 8 h, and 24 h). 300 μL of acetonitrile was added to it and vortexed followed by centrifugation at 15000 rpm for 15 min. The supernatant was taken and analyzed for the remaining amount of parent compound using RP-HPLC (Vydac 218TP C18 10μ, Length: 250 mm, ID: 4.6 mm). Each sample was run twice and each time in duplet.

### In Vivo Study

Animals: Adult male Sprague-Dawley rats (225-300 g; Harlan, Indianapolis, Ind.) and ICR mice (15-20 g; Harlan, Indianapolis, Ind.) were kept in a temperature-controlled environment with lights on 07:00-19:00 with food and water available ad libitum. All animal procedures were performed in accordance with the policies and recommendations of the International Association for the Study of Pain, the National Institutes of Health, and with approval from the Animal Care and Use Committee of the University of Arizona for the handling and use of laboratory animals.

Surgical methods: Rats were anesthetized (ketamine/xylazine anesthesia, 80/12 mg/kg i.p.; Sigma-Aldrich) and placed in a stereotaxic head holder. The cisterna magna was exposed and incised, and an 8-cm catheter (PE-10; Stoelting) was implanted as previously reported, terminating in the lumbar region of the spinal cord (Yaksh and Rudy, 1976). Catheters were sutured (3-0 silk suture) into the deep muscle and externalized at the back of the neck. After a recovery period (≥7 days) after implantation of the indwelling cannula, vehicle (10% DMSO: 90% MPH₂O) or AKG115 (0.1 μg; n=6/treatment) were injected in a 5 μL volume followed by a 9 μl saline flush. Catheter placement was verified at completion of experiments.

Behavioral Assay: Paw-flick latency [Hargreaves et al., 1988] was collected as follows. Rats were allowed to acclimate to the testing room for 30 minutes prior to testing. Basal paw withdrawal latencies (PWLs) to an infrared radiant heat source were measured (intensity=40) and ranged between 16.0 and 20.0 seconds. A cutoff time of 33.0 seconds was used to prevent tissue damage. After a single, intrathecal injection (i.t.) of AKG115 or vehicle, PWLs were re-assessed up to 8 times post-injection.

In follow-up studies with AKG127, we chose a mouse model of acute thermal pain (Tail flick latency—TFL) and administered our compound by lumbar puncture (Hylden and Wilcox, 1980) to eliminate the need for intrathecal catheters. Briefly, the latency to tail withdrawal (TFL) from a 52° C. water bath were measured before (baseline) intrathecal injection of AKG127 (0.1 μg in 5 μL volume, n=6-8/treatment). Tail flick latencies were re-assessed at up to 8 time points after administration. At cut-off latency of 10.0 s was implemented to prevent tissue damage to the distal third of the tail. Mice with baseline TFLs<3 s or >9 s were excluded from the study.

For both studies, maximal percent efficacy was calculated and expressed as:

$$\% Antinociception = \frac{( test latency after drug treatment - baseline latency )}{( cutoff - baseline latency )} \times 100 \%$$

Statistics: Between group data were analyzed by non-parametric two-way analysis of variance (ANOVA; post hoc: NeumanKuels) in FlashCalc (Dr. Michael H. Ossipov, University of Arizona, Tucson, Ariz., USA). Within group data were analyzed by non-parametric one-way analysis of variance (ANOVA; post hoc: Bonferroni) in FlashCalc (Dr. Michael H. Ossipov, University of Arizona, Tucson, Ariz., USA). Differences were considered to be significant if P≤0.05. All data were plotted in GraphPad Prism 6.

Compounds: AKG115 and AKG127 were prepared in 10% DMSO in 90% MPH₂O

### Result and Discussion

In vitro biological study: Investigation was directed to identify the multivalent/multifunctional ligands (i.e., oligopeptides) with different ratios of binding affinities and agonist activity at MOR and DOR while showing their high affinity and antagonist activity at NK1R. To achieve this, unnatural amino acids including Dmt (2,6-dimethyl tyrosine), D-alanine, N-methylated amino acids, 4-Abz, 4-Amb, 4-Apac, 4-Ampa, and chiral benzyl amine etc., were introduced to the oligopeptides or ligands.

In some embodiments, opioid and NK-1 pharmacophores were directly connected with each other without any linker. The main changes made during this study are the introduction of unnatural amino acids (e.g., Tyr and Phe derivatives), and N-methylated amino acids. Some examples of oligopeptides of the invention are provided in Table 1 below.

TABLE I
Representative Oligopeptides

| | |
|---|---|
| AKG117: | H-Tyr-D-Ala-Gly-NMePhe-Pro-Leu-Trp-NH-Bn |
| | (3′,5′-(CF₃)₂) (SEQ ID NO: 11); |
| AKG115: | H-Dmt-D-Ala-Gly-NMePhe-Pro-Leu-Trp-NH-Bn |
| | (3′,5′-(CF₃)₂) (SEQ ID NO: 12); |
| AKG116: | H-Dmt-D-Ala-Gly-Phe-Pro-Leu-Trp-NH-Bn |
| | (3′,5′-(CF₃)₂) (SEQ ID NO: 13); |
| AKG127: | H-Dmt-D-Ala-Gly-Phe(4-F)-Pro-Leu-Trp-NH- |
| | Bn(3′,5′-(CF₃)₂) (SEQ ID NO: 14); |
| AKG128: | H-Dmt-D-Ala-Gly-NMePhe(4-F)-Pro-Leu-Trp- |
| | NH-Bn(3′,5′-(CF₃)₂) (SEQ ID NO: 15); |
| AKG190: | H-Tyr-D-Ala-Gly-Phe(4-F)-Pro-Leu-Trp-NH- |
| | Bn(3′,5′-(CF₃)₂) (SEQ ID NO: 52); |
| AKG191: | H-Dmt-D-Ala-Gly-Phe(4-Cl)-Pro-Leu-Trp- |
| | NH-Bn(3′,5′-(CF₃)₂) (SEQ ID NO: 16); |
| AKG192: | H-Dmt-D-Ala-Gly-Phe(4-Br)-Pro-Leu-Trp- |
| | NH-Bn(3′,5′-(CF₃)₂) (SEQ ID NO: 17); |
| AKG193: | H-Dmt-D-Ala-Gly-Phe(44)-Pro-Leu-Trp-NH- |
| | Bn(3′,5′-(CF₃)₂) (SEQ ID NO: 18); |
| AKG180: | H-Dmt-D-Ala-NMeGly-Phe-Pro-Leu-Trp-NH- |
| | Bn(3′,5′- (CF₃)₂)(SEQ ID NO: 53); |
| AKG181: | H-Dmt-D-MeAla-Gly-Phe-Pro-Leu-Trp-NH- |
| | Bn(3′,5′-(CF₃)₂) (SEQ ID NO: 54); |
| AKG182: | H-Dmt-D-Ala-NMeGly-NMePhe-Pro-Leu-Trp- |
| | NH-Bn(3′,5′-(CF₃)₂) (SEQ ID NO: 55); |
| AKG183: | H-Dmt-D-NMeAla-NMeGly-Phe-Pro-Leu-Trp- |
| | NH-Bn(3′,5′-(CF₃)₂) (SEQ ID NO: 56); |
| AKG184: | H-Dmt-D-NMeAla-Gly-NMePhe-Pro-Leu-Trp- |
| | NH-Bn(3′,5′-(CF₃)₂) (SEQ ID NO: 57); |
| AKG185: | H-Dmt-D-NMeAla-NMeGly-NMePhe-Pro-Leu- |
| | Trp-NH-Bn(3′,5′-(CF₃)₂) (SEQ ID NO: 58). |

The present inventors have previous shown that when the linker between opioid and NK1 pharmacophores are removed from the ligand TY027, the resulting ligand TY012 (where Met⁵ is) became p-selective. Ligand AKG117 is produced by replacement of Phe at 4^{th} position of ligand TY012 by NMePhe. It showed 9 times binding selectivity for MOR over DOR receptors (Kᵢ^{μ}=27 nM, Kᵢ^{δ}=240 nM, Table II) while showing potent binding affinity at NK1 receptors (Kᵢ^{hNK1}=3.4 nM, Kᵢ^{rNK1}=61 nM, Table II) meaning no appreciable change in binding affinities while comparing those for TY012 (Kᵢ^{μ}=9.5 nM, Kᵢ^{δ}=72 nM, Kᵢ^{hNK1}=0.61 nM, Kᵢ^{rNK1}=33 nM, Table II). Functional assays with ligand AKG117 also showed no appreciable change in agonist activities at opioid receptors (IC₅₀^{μ}=230 nM, IC₅₀^{δ}=100 nM, Table III) compared to those for TY012 (IC₅₀^{μ}=350 nM, IC₅₀^{δ}=45 nM, Table III). So, introduction of NMePhe alone at 4^{th} position has minimum impact in altering the in vitro biological profiles. Dmt is well known to increase the binding affinities at opioid receptors.

The ligand AKG115, where Tyr at 1^{st} position of ligand AKG117 was replaced by Dmt, showed 5 times binding selectivity for MOR (Kᵢ^{μ}=1 nM, Kᵢ^{δ}=5 nM, Table IV) and slightly more agonist activity at MOR over DOR (IC₅₀^{μ}=21 nM, IC₅₀^{δ}=31 nM, Table V) while showing its high binding affinity and antagonist activity at NK1 receptor (Kᵢ^{hNK1}=2 nM, Kᵢ^{rNK1}=48.3 nM, Table II; Kₑ^{NK1}=9.7 nM, Table III). This indicates that presence of Dmt at 1^{st} position played a role in increasing binding affinities and agonist activities at μ/δ opioid receptors. To cross-check whether N-methylated Phe at 4^{th} position in AKG115 had any impact in binding affinities and functional activities, ligand AKG116 having Phe in place of NMePhe was designed and synthesized keeping Dmt at 1^{st} position. This ligand showed high but balanced binding affinities for both μ and δ opioid receptors (Kᵢ^{μ}=3 nM, Kᵢ^{δ}=1 nM, Table II). But, its functional assays showed 26 times less agonist activity at MOR compared to that at DOR (IC₅₀^{μ}=80.8 nM, IC₅₀^{δ}=3.1 nM, Table III). It produced slightly increased binding affinity but small decrease in antagonist activity at NK1R (Kᵢ^{hNK1}=1.4 nM, Kᵢ^{rNK1}=27 nM, Table II; Kₑ^{NK1}=25 nM, Table III).

From the results observed for ligands AKG117, AKG115 and AKG116 it is evident that presence of Dmt at 1^{st} position and N-methylated Phe at 4^{th} position is required for higher agonist activity at MOR than that at DOR. These results also are consistent with our previous observations that structural change at opioid pharmacophores can have impact in the biological profiles at NK1 receptors. Presence of halogens in drug candidates, especially in aromatic moieties, is known to play influential roles in their affinity and activities at biological targets. In ligands AKG127, AKG128, AKG190, AKG191, AKG192 and AKG193, we examined the effects of the presence of halogens. Though among halogen containing natural products, the presence of fluorine is less common, it has been found that presence of single or multiple fluorine atoms in synthetic drug candidates has profound effect in their biological profiles. In ligands AKG127, AKG128, and AKG190, we studied the effect of Phe(4-F) at 4^{th} position while carrying some local structural changes in the opioid pharmacophore. When we replaced the Phe from ligand AKG116 by 4-fluorophenylalanine i.e. Phe(4-F) to produce ligand AKG127, it showed balanced binding affinities at MOR and DOR (Kᵢ^{μ}=1 nM, Kᵢ^{δ}=1 nM, Table II) while showing high affinity for NK1 receptors (Kᵢ^{hNK1}=1 nM, Kᵢ^{rNK1}=29 nM, Table III). But the functional assay results showed 21 times selectivity for DOR over MOR while exerting high antagonist activity at NK1 receptor (IC₅₀^{μ}=42 nM, IC₅₀^{δ}=1.9 nM, Kₑ^{NK1}=5.3 nM, Table III). This might be due to the fact that all bonded ligands to MOR are not involved in its activation.

To check the effect of combination of N-methylation and presence of fluorine, the ligand AKG128, which contains N-methylated 4-fluorophenylalanine (NMe-Phe(4-F)) as its 4^{th} residue was synthesized and tested. It showed good binding affinity at all three receptors but with small selectivity for MOR over DOR (Kᵢ^{μ}=1 nM, Kᵢ^{δ}=4 nM, Kᵢ^{hNK1}=2.6 nM, Kᵢ^{rNK1}=34 nM, Table II). But functional assays showed nearly 7 times lower agonist activity at MOR than that at DOR while maintaining antagonist activity at NK1R (IC₅₀^{μ}=76.5 nM, IC₅₀^{δ}=11 nM, Kₑ^{NK1}=11 nM, Table III). Substitution of Dmt at Pt position by Tyr from ligand AKG127 gave ligand AKG190, which displayed selectivity for DOR over MOR in binding (20 times, Kᵢ^{μ}=4 nM, Kᵢ^{δ}=0.2 nM, Table II) as well as in functional assays (5 times, IC₅₀^{μ}=65 nM, IC₅₀^{δ}=12 nM, Table III).

To investigate the effect of other halogens ligands AKG191 containing Phe(4-C1), AKG192 containing Phe(4-Br), and AKG193 containing Phe(4-I) as 4^{th} residue were synthesized and tested. All of them showed reduced binding affinities (Table II) as well as functional activities (Table III) at opioid receptors compared to the parent ligand AKG127. But they displayed comparable binding affinities (Table II) as well as functional activities (Table III) at NK1 receptors. Iodine containing ligand AKG193 became much less active at the MOR though it showed good affinity at the same receptor. This again indicates binding of ligand to a receptor does not necessarily mean that it involves in functional activities.

To examine the effect of N-methylation at different residues as well as the impact of multiple N-methylations, different oligopeptides were designed and synthesized including, but not limited to, AKG180, AKG181, AKG182, AKG183, AKG184, and AKG185. Their partial in vitro results are given in Table II.

TABLE II
Binding affinity results of representative ligands at opioid and NK1 receptors

| Ligand No. | Kᵢ^{u} (nM) | Log[IC₅₀±] | Kᵢ^{δ} (nM) | Log[IC₅₀±] | Kᵢ^{u}/Kᵢ^{δ} | Kᵢ^{hNK1} (nm) | Kᵢ^{rNK1} (nm) | Kᵢ^{hNK1}/Kᵢ^{rNK1} |
|---|---|---|---|---|---|---|---|---|
| TY012 | 9.5 | −7.7 ± 0.21 | 72 | −6.8 ± 0.08 | 1/8 | 0.6 | 33 | 1/54 |
| AKG117 | 27 | −7.05 ± 0.04 | 237 | −6.35 ± 0.13 | 1/9 | 3.35 ± 0.74 | 61.1 ± 2.0 | 1/18 |
| | (n = 6) | | (n = 6) | | | (n = 6) | (n = 6) | |
| AKG115 | 1 | −8.78 ± 0.05 | 5 | −7.92 ± 0.07 | 1/5 | 2.23 ± 0.07 | 48.3 ± 8.32 | 1/22 |
| | (n = 6) | | (n = 6) | | | (n = 6) | (n = 6) | |
| AKG116 | 3 | −8.63 ± 0.04 | 1 | −8.66 ± 0.03 | 3/1 | 1.4 ± 0.09 | 26.9 ± 1.98 | 1/19 |
| | (n = 6) | | (n = 6) | | | (n = 6) | (n = 6) | |
| AKG127 | 1 | −8.72 ± 0.08 | 1 | −7.18 ± 0.04 | 1/1 | 0.88 ± 0.07 | 29.4 ± 1.5 | 1/33 |
| | (n = 6) | | (n = 6) | | | (n = 6) | (n = 6) | |
| AKG128 | 1 | −8.55 ± 0.18 | 4 | −8.19 ± 0.08 | 1/4 | 2.62 ± 0.51 | 33.8 ± 6.2 | 1/13 |
| | (n = 2) | | (n = 6) | | | (n = 6) | (n = 6) | |
| AKG190 | 4 | −8.08 ± 0.10 | 0.2 | 7.65 ± 0.07 | 20/1 | 5.61 ± 0.65 | 34.4 ± 2.8 | 1/6 |
| | (n = 2) | | (n = 2) | | | (n = 6) | (n = 6) | |
| AKG191 | 2 | −8.33 ± 0.09 | 5 | −8.02 ± 0.03 | 1/2.5 | 2.9 ± 0.53 | 26.5 ± 5.3 | 1/9 |
| | (n = 2) | | (n = 6) | | | (n = 6) | (n = 6) | |
| AKG192 | 5 | −7.96 ± 0.12 | 16 | −7.71 ± 0.06 | 1/2 | 2.54 ± 0.21 | 47.4 ± 12.6 | 1/19 |
| | (n = 2) | | (n = 4) | | | (n = 6) | (n = 6) | |
| AKG193 | 6 | −7.88 ± 0.07 | 10 | −7.53 ± 0.08 | 1/3 | 3.29 ± 0.6 | 38.8 ± 3.6 | 1/12 |
| | (n = 2) | | (n = 4) | | | (n = 6) | (n = 6) | |
| AKG180 | N.D. | N.D. | N.D. | N.D. | -/- | 2.86 | 17.1 ± 2.9 | 1/6 |
| | | | | | | (n = 4) | (n = 6) | |
| AKG181 | N.D. | N.D. | N.D. | N.D. | -/- | 4.81 ± 1.39 | 26.4 ± 9.3 | 1/5 |
| | | | | | | (n = 6) | (n = 6) | |
| AKG182 | N.D. | N.D. | N.D. | N.D. | -/- | 5.00 | 110.0 ± 18.3 | 1/22 |
| | | | | | | (n = 4) | (n = 6) | |
| AKG183 | N.D. | N.D. | N.D. | N.D. | -/- | 2.49 | 17.6 ± 10. | 1/7 |
| | | | | | | (n = 4) | (n = 6) | |
| AKG184 | N.D. | N.D. | N.D. | N.D. | -/- | 2.84 | 114.8 | 1/40 |
| | | | | | | (n = 4) | (n = 4) | |
| AKG185 | N.D. | N.D. | N.D. | N.D. | -/- | 3.47 | 74.83 | 1/21 |
| | | | | | | (n = 4) | (n = 4) | |

N.D. means not determined,
n in the parenthesis indicates number of run

TABLE III
Functional assay results of representative ligands

| Compd. | GPI (MOR) | MVD (DOR) | GPI/MVD | GPI/LMMP (NK1R) | |
|---|---|---|---|---|---|
| Number | IC₅₀ (nM) | IC₅₀ (nM) | IC₅₀ ratio | Agonist | Kₑ (nM) ± S.E.M. |
| AKG117 | 231.7 ± 52.9 | 102.5 ± 33.6 | 2.3/1 | None at 100 nM | 21.1 ± 9.2 |
| AKG115 | 20.6 ± 3.52 | 30.7 ± 7.5 | 1/1.5 | None at 30 nM | 9.7 ± 1.2 |
| AKG116 | 80.8 ± 18.1 | 3.1 ± 1.0 | 26/1 | None at 100 nM | 24.9 ± 3.6 |
| AKG127 | 41.6 ± 9.68 | 1.96 ± 0.680 | 21.2/1 | None at 30 nM | 5.3 ± 1.64 |
| AKG128 | 76.5 ± 14.96 | 11.5 ± 5.6 | 6.6/2 | None at 30 nM | 11.2 ± 2.7 |
| AKG190 | 64.8 ± 9.2 | 12.1 ± 4.0 | 5.3/1 | None at 30 nM | 5.8 ± 1.9 |
| AKG191 | 166.2 ± 71.6 | 25.4 ± 7.7 | 6.5/1 | None at 300 nM | 44.1 ± 7.7 |
| AKG192 | 463.4 ± 114.3 | 43.0 ± 11.6 | 10.8/1 | None at 100 nM | 23.4 ± 8.9 |
| AKG193 | 41% at 1 uM | 97.2 ± 20.5 | —/— | None at 300 nM | 41.9 ± 5.9 |

For every sample, the number of run was four at each receptor

In other embodiments, a wide variety of natural and unnatural amino acids that have been incorporated as a linker and/or an address region were prepared and tested. See Table IV. For the first time, aromatic rigid linkers, e.g., 4-Amb, 4-Abz, 4-Apac, etc., have been introduced to reduce the interference of opioid and NK-1 pharmacophores in each other's activity. N-methylated unnatural amino acids also were used during this study.

TABLE IV
Other Representative Oligopeptides of the
Invention

| | |
|---|---|
| TY045: | H-Tyr-D-Ala-Gly-Phe-Nle-Pro-Leu-Trp-NH- |
| | Bn(3′,5′-(CF₃)₂) (SEQ ID NO: 59); |
| KG112: | H-Tyr-D-Ala-Gly-NMePhe-Nle-Pro-Leu-Trp- |
| | NH-Bn(3′,5′-(CF₃)₂) (SEQ ID NO: 60); |
| AKG113: | H-Tyr-D-Ala-Gly-NMePhe-Gly-Pro-Leu-Trp- |
| | NH-Bn(3′,5′-(CF₃)₂) (SEQ ID NO: 61); |
| AKG130: | H-Tyr-D-Ala-Gly-Phe(4-F)-Gly-Pro-Leu-Trp- |
| | NH-Bn(3′,5′-(CF₃)₂) (SEQ ID NO: 62); |
| AKG131: | (SEQ ID NO: 10, where Tyr′ = Tyr, R on |
| | Ala is methyl, Phe′ is NMePhe(4-F), |
| | x = 1, AA is Gly, R on Leu, Trp is H, and |
| | -XCH(R)-Ph(R′)₂ is -NHCH2(3,5- |
| | difluorophenyl); |
| AKG119: | (SEQ ID NO: 19, where Tyr′ = Tyr, R on |
| | Ala, Gly, Leu, and Trp is H, Phe′ = |
| | NMePhe, x = 1, AA = β-Ala, and -XCH(R)- |
| | Ph(R′)₂ is -NHCH₂(3,5-difluorophenyl); |
| AKG123: | (same as AKG119 except AA = 4-Abu); |
| AKG124: | (same as AKG119 except AA = 6-Ahx); |
| AKG125: | (same as AKG119 except AA = 4-Amb); |
| AKG176: | (same as AKG119 except AA = 4-Abz); |
| AKG177: | (same as AKG119 except AA = 4-Apac); |
| AKG178: | (same as AKG119 except AA = 4-Ampa); |
| AKG179: | (same as AKG119 except Tyr′ is Dmt and |
| | AA = 4-Abu); |
| AKG106: | (same as AKG119 except AA = Met); |
| AKG107: | (same as AKG119 except Tyr′ = Dmt and |
| | AA = Met). |

This study was started taking ligand TY045, which showed selectivity for MOR over DOR, as a reference. As the biasedness for MOR was observed because of the introduction of NMe-Phe at the 4^{th} position, the Phe was replaced by the same to get more selectivity for MOR. Ligand AKG112 containing NMe-Phe as 4^{th} residue and Nle as 5^{th} residue showed small binding selectivity (1.7 time) for MOR over DOR (Kᵢ^{μ}=13 nM, Kᵢ^{δ}=22 nM, Table V) while showing nanomolar range binding affinity at NK1R (Kᵢ^{hNK1}=3.8 nM, Kᵢ^{rNK1}=19 nM, Table V). However, functional activity studies showed that it has higher agonist activity at DOR compared to that at MOR (IC₅₀^{μ}=718.5 nM, IC₅₀^{δ}=12 nM, Kₑ^{NK1}=5.4 nM, Table V).

In search of further increase of p-selectivity, substitution of Nle from AKG112 by relatively flexible Gly was made to produce ligand AKG113. This ligand exhibited 15 times binding selectivity (Kᵢ^{μ}=3 nM, Kᵢ^{δ}=46 nM, Table V) but with small reduction in binding affinity at rat NK1 (Kᵢ^{hNK1}=2.1 nM, Kᵢ^{rNK1}=60 nM, Table V). Functional assays showed that it has two times higher agonist activity at DOR compared to that at MOR while exhibiting high antagonist activity at NK1R (IC₅₀^{μ}=79.20 nM, IC₅₀^{δ}=39 nM, Kₑ^{NK1}=15 nM, Table V). To examine the effect of fluorine, ligands AKG130 and AKG131, which have different 4^{th} residue of AKG113, i.e. Phe by Phe(4-F) and NMePhe(4-F), respectively, were synthesized. Both the ligands showed binding selectivity for DOR binding (AKG130: Kᵢ^{μ}=9 nM, Kᵢ^{δ}=5 nM; AKG131: Kᵢ^{μ}=185 nM, Kᵢ^{δ}=17 nM, Table V) with reduced binding affinity at rat NK1R (AKG130: Kᵢ^{hNK1}=1.54 nM, Kᵢ^{rNK1}=72 nM; AKG131: Kᵢ^{hNK1}=2 nM, Kᵢ^{rNK1}=89 nM, Table V). Both of these ligands displayed higher agonist activity at DOR compared to that at DOR NK1R (AKG130: IC₅₀^{μ}=340 nM, IC₅₀^{δ}=12 nM, Kₑ^{NK1}=11 nM; AKG131: IC₅₀^{μ}=63 nM, IC₅₀^{δ}=30 nM, Kₑ^{NK1}=36 nM; Table V).

To examine the impact of longer and flexible linker/address region, ligands AKG119, AKG123 and AKG124, which are the products of the substitution of 5^{th} residue of AKG113 i.e., Gly by β-Ala, γ-Abu and 6-Ahx, respectively, were synthesized. All these ligands showed binding affinities at nanomolar range. However, no significant increase in opioid receptor binding selectivity was found (Table V). Functional assays showed 7-10 times higher agonist activity at DOR compared to that at MOR (Table V). It was noticed that modification of opioid pharmacophore was impacting binding affinities at opioid receptors as well as at NK1R. At this point it was thought that introduction of a rigid linker in between opioid and NK1 receptors might reduce the interference of each receptor in other's biological profile. The 5^{th} residue Gly from AKG113 was replaced with relatively rigid linker 4-Amb to obtain ligand AKG125. This new ligand exhibited 23 times binding selectivity for MOR over DOR (Kᵢ^{μ}=5 nM, Kᵢ^{δ}=120 nM, Table V) while maintaining nanomolar affinity at NK1 receptors (Kᵢ^{hNK1}=2.3 nM, Kᵢ^{rNK1}=30 nM, Table V). However, it produced 10 times higher agonist activity at DOR (IC₅₀^{μ}=470 nM, IC₅₀^{δ}=46 nM, Kₑ^{NK1}=8.4 nM, Table V).

Based at least in part on this result, ligands AKG176, AKG177 and AKG178 containing 4-Abz, 4-Apac and 4-Ampa as linkers, respectively, were synthesized. Ligand AKG176 having the most rigid linker showed higher affinity at MOR (Kᵢ^{μ}=1 nM, Kᵢ^{δ}=70 nM, Table V) while maintaining good NK1R binding affinity (Kᵢ^{hNK1}=5.3 nM, Kᵢ^{rNK1}=74 nM, Table V). Ligand AKG177 displayed almost equal agonist activity at MOR and DOR (IC₅₀^{μ}=41 nM, IC₅₀^{δ}=35 nM, Table V) while showing high antagonist activity at NK1R (Kₑ^{NK1}=39 nM, Table V). Ligand AKG178, which has an address region moiety of aromatic rigidity in the middle with two flexible arms at 180 degree angle, have shown high binding affinity DOR (Kᵢ^{δ}=100 nM, Table IV) as well as NK1 receptors (Kᵢ^{hNK1}=3.5 nM, Kᵢ^{rNK1}=72 nM, Table IV). But it showed 15 times higher agonist activity at DOR in functional assays (IC₅₀^{μ}=650 nM, IC₅₀^{δ}=44 nM, Kₑ^{NK1}=76 nM, Table V). AKG179 was designed and synthesized by replacing the Tyr by Dmt position to increase the binding affinities and functional activities at opioid receptors. In addition, ligands AKG106 and AKG107 were designed and synthesized by replacing 4^{th} residue i.e. Phe of TY027 (H-Tyr-D-Ala-Gly-Phe-Met-Pro-Leu-Trp-NH-Bn(3′,5″-(CF₃)₂)) and TY032 (H-Dmt-D-Ala-Gly-Phe-Met-Pro-Leu-Trp-NH-Bn(3′,5″-(CF₃)₂)) by NMePhe. These ligands have shown high binding affinities at NK1R (TY027: Kᵢ^{hNK1}=1.5 nM, Kᵢ^{rNK1}=10 nM; TY027: Kᵢ^{hNK1}=2 nM, Kᵢ^{rNK1}=14 nM).

TABLE V
Binding affinity results at opioid and NK1 receptors

| Ligand No. | Kᵢ^{u} (nM) | Log[IC₅₀±] | Kᵢ^{δ} (nM) | Log[IC₅₀±] | Kᵢ^{u}/Kᵢ^{δ} | Kᵢ^{hNK1} (nm) | Kᵢ^{rNK1} (nm) | Kᵢ^{hNK1}/Kᵢ^{rNK1} |
|---|---|---|---|---|---|---|---|---|
| AKG112 | 13 | −7.51 ± 0.04 | 22 | −7.30 ± 0.05 | 1/1.7 | 14.0 ± 3. | 19.12 ± 7.88 | 1/1.4 |
| | (n = 2) | | (n = 2) | | | (n = 6) 1 | | |
| AKG113 | 3 | −8.23 ± 0.03 | 46 | −7.00 ± 0.06 | 1/15 | 15.0 ± 4.06 | 60.5 ± 2.9 | 1/4 |
| | (n = 2) | | (n = 2) | | | (n = 6) | (n = 6) | |
| AKG130 | 9 | −7.69 ± 0.06 | 5 | −7.96 ± 0.04 | 2/1 | 1.54 ± 0.12 | 72.5 ± 10.0 | 1/47 |
| | (n = 2) | | (n = 2) | | | (n = 6) | (n = 6) | |
| AKG131 | 185 | −6.40 ± 0.32 | 17 | −7.42 ± 0.07 | 11/1 | 2.14 ± 0.31 | 89.1 ± 11.6 | 1/8 |
| | (n = 2) | | (n = 2) | | | (n = 6) | (n = 6) | |
| AKG119 | 8 | −7.76 ± 0.11 | 46 | −6.97 ± 0.05 | 1/5.6 | 0.93 ± 0.14 | 29.43 ± 2.66 | 1/32 |
| | (n = 2) | | (n = 2) | | | (n = 6) | (n = 6) | |
| AKG123 | 9 | −7.73 ± 0.07 | 3 | −8.23 ± 0.05 | 3/1 | 1.56 ± 0.27 | 57.0 ± 1.9 | 1/37 |
| | (n = 2) | | (n = 2) | | | (n = 6) | (n = 6) | |
| AKG124 | 7 | −7.80 ± 0.09 | 31 | −7.14 ± 0.03 | 1/4.4 | 1.22 ± 0.3 | 44.3 ± 2.9 | 1/36 |
| | (n = 2) | | (n = 2) | | | (n = 6) | (n = 6) | |
| AKG125 | 5 | −7.92 ± 0.11 | 117 | −6.58 ± 0.06 | 1/23.4 | 2.27 ± 0.68 | 29.9 ± 7.6 | 1/13 |
| | (n = 2) | | (11 = 2) | | | (n = 6) | (n = 6) | |
| AKG176 | 1 | −8.69 ± 0.24 | 82 | −6.74 ± 0.16 | 1/82 | 4.12 ± 0.94 | 77.8 ± 4.8 | 1/19 |
| | (n = 2) | | (n = 6) | | | (n = 6) | (n = 6) | |
| AKG177 | 1 | −8.68 ± 0.19 | 70 | −6.83 ± 0.06 | 1/70 | 5.28 ± 1.15 | 74.3 ± 12.07 | 1/14 |
| | (n = 2) | | (n = 4) | | | (n = 6) | (n = 6) | |
| AKG178 | N.D. | N.D. | 100 | −6.64 ± 0.17 | -/- | 3.50 ± 1.05 | 72.3 ± 11.2 | 1/21 |
| | | | (n = 4) | | | (n = 6) | (n = 6) | |
| AKG106 | N.D. | N.D. | N.D. | N.D. | -/- | 1.47 ± 1.8 | 9.9 ± 2.3 | 1/7 |
| | | | | | | (n = 6) | (n = 6) | |
| AKG107 | N.D. | N.D. | N.D. | N.D. | -/- | 1.97 ± 2.6 | 13.6 ± 1.4 | 1/7 |
| | | | | | | (n = 6) | (n = 6) | |

N.D. means not determined,
n in the parenthesis indicates number of run

TABLE VI
Functional assay results

| Compd. | GPI (MOR) | MVD (DOR) | GPI/MVD | GPI/LMMP (NK1R) | |
|---|---|---|---|---|---|
| Number | IC₅₀ (nM) | IC₅₀ (nM) | IC₅₀ ratio | Agonist | Kₑ (nM) ± S.E.M. |
| AKG112 | 718.5 ± 168.7 | 11.63 ± 2.55 | 62/1 | None at 30 nM | 5.4 ± 2.1 |
| AKG113 | 79.20 ± 5.52 | 38.74 ± 9.69 | 2/1 | None at 100 nM | 14.9 ± 2.9 |
| AKG130 | 339.4 ± 120.0 | 11.91 ± 1.56 | 28.5/1 | None at 300 nM | 10.6 ± 3.54 |
| AKG131 | 62.55 ± 19.29 | 29.71 ± 9.73 | 2/1 | None at 100 nM | 35.8 ± 10.8 |
| AKG119 | 365.4 ± 185.5 | 36.67 ± 4.18 | 10/1 | None at 100 nM | 20.8 ± 3.8 |
| AKG123 | 423.6 ± 147.8 | 50.08 ± 17.43 | 8.5/1 | None at 100 nM | 2.3 ± 0.7 |
| AKG124 | 254.7 ± 71.2 | 36.82 ± 13.59 | 7/1 | None at 100 nM | 11.2 ± 3.1 |
| AKG125 | 471.1 ± 273.2 | 46.26 ± 13.42 | 10/1 | None at 100 nM | 8.36 ± 4.12 |
| AKG176 | 40.79 ± 8.00 | 22.78 ± 5.53 | 2/1 | None at 300 nM | 116.6 ± 31.4 |
| AKG177 | 40.82 ± 6.00 | 35.01 ± 7.89 | 1/1 | None at 100 nM | 39.2 ± 7.9 |
| AKG178 | 654.0 ± 76.7 | 44.02 ± 16.00 | 15/1 | None at 300 nM | 76.4 ± 11.2 |

For every sample, the number of run was four at each receptor

In other embodiments, DAMGO (H-Tyr-D-Ala-Gly-NMePhe-Gly-ol) derived pharmacophores are incorporated in the opioid part of the new ligands. The side chain of the 5^{th} residue contained functional groups like free alcoholic hydroxyl (—OH), and amine (—NH₂). N-methylated unnatural amino acids have been used during this study.

TABLE VII
Further representative Oligopeptides

| | |
|---|---|
| AKG038: | (same as AKG119 except AA = Ser); |
| AKG039: | (same as AKG119 except Phe′ = Phe and AA = |
| | Homo-Ser); (same as AKG119 except |
| | Phe′ = Phe, AA = D-Ser); |
| AKG126: | (same as AKG119 except AA = Ser); |
| AKG132: | (same as AKG119 except Tyr′ = Dmt and AA = Ser); |
| AKG133: | (same as AKG119 except Phe′ = Phe(4-F) |
| | and AA = Ser); |
| AKG134: | (same as AKG119 except Phe′ = NMePhe(4-F) |
| | and AA = Ser); |
| AKG135: | (same as AKG119 except Tyr′ = Dmt, Phe′ = |
| | NMePhe(4-F), and AA = Ser); |
| AKG-CRA-136: | (same as AKG119 except x = 2 and (AA)ₓ = Ser-Gly; |
| AKG-CRA-137: | (same as AKG119 except Tyr′ = Dmt, |
| | x = 2 and (AA)ₓ = Ser-Gly); |
| AKG171: | (same as AKG119 except AA = 4-Dap); |
| AKG172: | (same as AKG119 except AA = 4-Dab); |
| AKG173: | (same as AKG119 except AA = Orn); |
| AKG174: | (same as AKG119 except AA = Lys). |

Some ligands showed higher agonist activities DOR compared to that at MOR. In some cases, it would be beneficial to achieve higher binding as well as functional selectivity at MOR compared to those at DOR. The structural features of DAMGO (H-Tyr-D-Ala-Gly-NMe-Phe-Gly-ol), a MOR selective ligand, was introduced in some of the oligopeptides of the invention. Ligand AKG038 was designed and synthesized by introducing serine (Ser) at the 5^{th} position (Table VIII). It was expected to play the role similar to that played by glyol (Gly-ol) in DAMGO. This ligand showed 18 times higher binding affinity at DOR compared to that at MOR Kᵢ^{μ}=130 nM, Kᵢ^{δ}=7 nM, Table VIII). This ligand showed low binding affinity at rNK1R (Kᵢ^{hNK1}=2 nM, Kᵢ^{rNK1}=210 nM, Table VIII). Functional assays showed 21 times higher agonist activity at DOR over DOR (IC₅₀^{μ}=400 nM, IC₅₀^{δ}=18 nM, Kₑ^{NK1}=5 nM, Table IX).

The effect of length of the side chain containing primary alcoholic group at 5^{th} position was also examined by replacing Ser with homo-serine (Homo-Ser) at 5^{th} position, e.g., ligand AKG039. There was no significant change in binding affinities at opioid receptors as well as NK1R (Kᵢ^{μ}=120 nM, Kᵢ^{δ}=6 nM, Kᵢ^{hNK1}=1.3 nM, Kᵢ^{rNK1}=150 nM, Table VIII). It also displayed 21 times higher agonist activity at DOR (IC₅₀^{μ}=130 nM, IC₅₀^{δ}=6 nM, Kₑ^{NK1}=9.7 nM, Table IX). Though these two ligands showed nanomolar range binding affinities at human NK1R, poor binding affinities were observed at rat NK1R.

Effect of chirality at 5^{th} position was checked by introducing D-Ser in ligand AKG101 (Table VIII). It did not significantly improve the binding affinities at MOR, DOR and NK1R (Kᵢ^{μ}=200 nM, Kᵢ^{δ}=34 nM, Kᵢ^{hNK1}=3 nM, Kᵢ^{rNK1}=110 nM, Table VIII). Functional assays showed that it had poor agonist activity at MOR (IC₅₀^{μ}=39.7% at 1 μM, IC₅₀^{δ}=6.7 nM, Kₑ^{NK1}=28 nM, Table IX). Replacement of Phe (of AKG038) by NMePhe produced ligand AKG126, which displayed 31 times binding selectivity for MOR over DOR while showing high affinity at NK1R (Kᵢ^{μ}=2 nM, Kᵢ^{δ}=63 nM, Kᵢ^{hNK1}=1 nM, Kᵢ^{rNK1}=31 nM, Table VIII). But it displayed 9 times higher agonist activity at DOR over MOR (IC₅₀^{μ}=240 nM, IC₅₀^{δ}=26 nM, Kₑ^{NK1}=17 nM, Table IX).

It was observed that the presence of Dmt at 1^{st} position of opioid ligands significantly increases the binding affinity. Ligand AKG132 was designed and synthesized by introducing Dmt at 1^{st} position. It showed the expected higher affinity at opioid receptors but with reduced binding selectivity (Kᵢ^{μ}=0.4 nM, Kᵢ^{δ}=2 nM, Kᵢ^{hNK1}=5.6 nM, Kᵢ^{rNK1}=36 nM, Table VIII). It also displayed delta selectivity over mu (IC₅₀^{μ}=43 nM, IC₅₀^{δ}=7.7 nM, Kₑ^{NK1}=20 nM, Table IX). Ligands AKG133, AKG134 and AKG135 were designed and synthesized using AKG038, AKG126 and AKG132 as references, respectively.

The effect of fluorine (F) in the para position of Phe (Table VII) was also studied. All of them showed high binding affinities (nanomolar range) at all three receptors (Table VIII). However, they failed to produce appreciable binding selectivity. All these three ligands displayed higher agonist activity at DOR compared to that at MOR with strong antagonist activity at NK1R (Table IX). However, ligand AKG135 showed high and close agonist activity at both the opioid receptors studied (IC₅₀^{μ}=23 nM, IC₅₀^{δ}=15 nM, KₑNK1=29 nM, Table VI). To examine the effect of the length of linker, Gly as 6^{th} residue was introduced in ligands AKG-CRA-136 and AKG-CRA-137 (Table VII). Ligand AKG-CRA-137 showed balanced binding affinities at MOR and DOR while showing good affinity at NK1R (Kᵢ^{μ}=0.7 nM, Kᵢ^{δ}=1 nM, Kᵢ^{hNK1}=4.9 nM, Kᵢ^{rNK1}=87 nM, Table VIII). This ligand having Dmt at Pt position and Gly at 6^{th} position showed two times higher agonist activity at MOR compared to that at DOR (IC₅₀^{μ}=7.9 nM, IC₅₀^{δ}=16 nM, Kₑ^{NK1}=26 nM, Table IX).

Ligands AKG171, AKG172, AKG173, and AKG174, were designed and synthesized by replacing 5^{th} residue i.e. Ser of AKG126 by Dap, Dab, Orn, and Lys respectively. Ligands AKG171 and AKG172 showed binding selectivity at MOR over DOR (AKG171: Kᵢ^{μ}=3 nM, Kᵢ^{δ}=33 nM, Kᵢ^{hNK1}=3 nM, Kᵢ^{rNK1}=8.5 nM; AKG172: Kᵢ^{μ}=8 nM, Kᵢ^{δ}=100 nM, Kᵢ^{hNK1}=3.05 nM, Kᵢ^{rNK1}=8.2 nM; Table VIII). Surprisingly, AKG173 containing Orn as 5^{th} residue showed 100 times binding selectivity at DOR over MOR (Kᵢ^{μ}=5 nM, Kᵢ^{δ}=0.05 nM, Kᵢ^{hNK1}=15.5 nM, Kᵢ^{rNK1}=65 nM, Table VIII). All these ligands showed strong binding affinity at NK1R and the difference between hNK1R and rNK1R binding affinities became low (Table VIII). Functional assays with these ligands are in progress.

TABLE VIII
Binding affinity results at opioid and NK1 receptors

| Ligand No. | Kᵢ^{u} (nM) | Log[IC₅₀±] | Kᵢ^{δ} (nM) | Log[IC₅₀±] | Kᵢ^{u}/Kᵢ^{δ} | Kᵢ^{hNK1} (nm) | Kᵢ^{rNK1} (nm) | Kᵢ^{hNK1}/Kᵢ^{rNK1} |
|---|---|---|---|---|---|---|---|---|
| AKG038 | 127 | −6.60 ± 0.12 | 7 | −7.74 ± 0.15 | 18/1 | 1.94 ± 0.25 | 206.1 ± 14.7 | 1/106 |
| | (n = 2) | | (n = 6) | | | | | |
| AKG039 | 116 | −6.66 ± 0.06 | 6 | −7.82 ± 0.28 | 19/1 | 1.32 ± 0.01 | 148.1 ± 9.8 | 1/112 |
| | (n = 2) | | (n = 6) | | | | | |
| AKG101 | 196 | −6.35 ± 0.26 | 34 | −7.13 ± 0.07 | 6/1 | 2.77 ± 0.51 | 108 ± 22.6 | 1/39 |
| | (n = 2) | | (n = 2) | | | | | |
| AKG126 | 2 | −8.30 ± 0.11 | 63 | −6.83 ± 0.05 | 1/31 | 0.86 ± 0.07 | 31.5 ± 6.64 | 1/37 |
| | (n = 2) | | (n = 2) | | | | | |
| AKG132 | 0.4 | −9.03 ± 0.06 | 2 | −8.28 ± 0.07 | 1/5 | 5.61 ± 0.65 | 31.1 ± 5.7 | 1/6 |
| | (n = 2) | | (n = 2) | | | | | |
| AKG133 | 8 | −7.69 ± 0.09 | 2 | −8.44 ± 0.03 | 4/1 | 4.86 ± 1.88 | 125.0 ± 40.5 | 1/26 |
| | (n = 2) | | (n = 2) | | | | | |
| AKG134 | 2 | −8.27 ± 0.11 | 7 | −7.84 ± 0.17 | 1/4 | 7.95 ± 0.87 | 50.2 ± 16.9 | 1/6 |
| | (n = 2) | | (n = 2) | | | | | |
| AKG135 | 0.5 | −8.93 ± 0.09 | 0.5 | −9.00 ± 0.03 | 1/1 | 7.26 ± 0.59 | 51.7 ± 16.2 | 1/7 |
| | (n = 2) | | (n = 2) | | | | | |
| AKG-CRA-136 | 3 | −8.07 ± 0.18 | 38 | −7.07 ± 0.08 | 1/9 | 9.23 ± 1.38 | 156.0 ± 2.33 | 1/17 |
| | (n = 4) | | (n = 4) | | | | | |
| AKG-CRA-137 | 0.7 | −8.82 ± 0.26 | 1 | −8.57 ± 0.07 | 1/1.4 | 4.76 ± 0.23 | 87.5 | 1/18 |
| | (n = 2) | | (n = 2) | | | | | |
| AKG171 | 3 | −8.24 ± 0.49 | 33 | −7.13 ± 0.05 | 1/11 | 2.98 ± 0.36 | 8.59 ± 0.89 | 1/3 |
| | (n = 2) | | (n = 2) | | | | | |
| AKG172 | 8 | −7.84 ± 0.11 | 100 | −6.65 ± 0.08 | 1/12 | 3.05 ± 0.33 | 8.24 ± 3.56 | 1/2.7 |
| AKG173 | 5 | −7.95 ± 0.08 | 0.05 | −6.30 ± 0.07 | 100/1 | 15.5 ± 2.03 | 65.2 ± 9.0 | 1/4.2 |
| AKG174 | 8 | −7.81 ± 0.08 | N.D. | N.D. | -/- | 3.18 ± 1.18 | 7.31 ± 0.89 | 1/2.3 |

N.D. means not determined,
n in the parenthesis indicates number of run

TABLE IX
Functional assay results

| Compd. | GPI (MOR) | MVD (DOR) | GPI/MVD | GPI/LMMP (NK1R) | |
|---|---|---|---|---|---|
| Number | IC₅₀ (nM) | IC₅₀ (nM) | IC₅₀ ratio | Agonist | Kₑ (nM) ± S.E.M. |
| AKG038 | 398.7 ± 107.9 | 18.41 ± 4.37 | 21.1/1 | None at 30 nM | 4.8 ± 1.6 |
| AKG039 | 128.5 ± 20.6 | 5.988 ± 1.346 | 21.5/1 | None at 100 nM | 9.7 ± 1.3 |
| AKG101 | 39.7% at 1 uM | 6.721 ± 1.931 | —/— | None at 100 nM | 28.0 ± 11.8 |
| AKG126 | 237.3 ± 29.2 | 25.89 ± 6.69 | 9/1 | None at 30 nM | 16.53 ± 7.77 |
| AKG132 | 42.65 ± 6.00 | 7.657 ± 2.033 | 5.6/1 | None at 30 nM | 19.8 ± 2.9 |
| AKG133 | 85.40 ± 18.71 | 10.95 ± 2.50 | 7.8/1 | None at 30 nM | 2.43 ± 0.72 |
| AKG134 | 184.4 ± 30.9 | 8.936 ± 0.795 | 21/1 | None at 30 nM | 1.86 ± 0.43 |
| AKG135 | 23.45 ± 4.74 | 15.40 ± 5.40 | 2/1 | None at 100 nM | 28.7 ± 11.1 |
| AKG-CRA-136 | 108.1 ± 44.8 | 22.62 ± 2.07 | 5/1 | None at 100 nM | 27.6 ± 2.9 |
| AKG-CRA-137 | 7.871 ± 2.567 | 16.39 ± 5.17 | 1/2 | None at 100 nM | 26.3 ± 5.7 |

For every sample, the number of run was four at each receptor

In some oligopeptides of the invention, amide (—CONH₂) functionality is incorporated in the side chain of 5^{th} amino acid residue, and N-methylated amino acids.

TABLE X
Further Representative Oligopeptides of the Invention

| | |
|---|---|
| AKG104: | (same as AKG119 except Phe′ = Phe and AA = Asn); |
| AKG102: | (same as AKG119 except Phe′ = Phe and |
| | AA = 4-D-Asn); |
| AKG105: | (same as AKG119 except Phe′ = Phe and AA = Gln); |
| AKG103: | (same as AKG119 except Phe′ = Phe and AA = D-Gln); |
| AKG129: | (same as AKG119 except AA = Gln); |
| AKG141: | (same as AKG119 except Tyr′ = Dmt and AA = Gln); |
| AKG142: | (same as AKG119 except Phe′ = Phe(4-F) and |
| | AA = Gln); |
| AKG143: | (same as AKG119 except Phe′ = NMePhe(4-F) |
| | and AA = Gln); |
| AKG144: | (same as AKG119 except Tyr′ = Dmt, Phe′ = |
| | NMePhe(4-F) and AA = Gln); |
| AKG-SK-145: | (same as AKG119 except x = 2 and (AA)x = Gln-Gly); |
| AKG-SK-146: | (same as AKG119 except Tyr′ = Dmt, x = 2, |
| | and (AA)x is Gln-Gly). |

TABLE XI
Binding affinity results at opioid and NK1 receptors

| Ligand No. | Kᵢ^{u} (nM) | Log[IC₅₀±] | Kᵢ^{δ} (nM) | Log[IC₅₀±] | Kᵢ^{u}/Kᵢ^{δ} | Kᵢ^{hNK1} (nm) | Kᵢ^{rNK1} (nm) | Kᵢ^{hNK1}/Kᵢ^{rNK1} |
|---|---|---|---|---|---|---|---|---|
| AKG104 | 51 | −6.93 ± 0.05 | 17 | 7.43 ± 0.08 | 3/1 | 1.16 ± 0.04 | 20.2 ± 0.73 | 1/17.4 |
| AKG102 | 90 | −6.68 ± 0.09 | 54 | −6.90 ± 0.06 | 1.7/1 | 2.26 ± 0.32 | 133 ± 3.5 | 1/58.8 |
| AKG105 | 38 | −7.06 ± 0.18 | 9 | −7.69 ± 0.13 | 4.2/1 | 0.92 ± 0.14 | 12.5 ± 1.3 | 1/13.6 |
| AKG103 | 116 | −6.57 ± 0.05 | 72 | −6.81 ± 0.07 | 1.6/1 | 2.71 ± 0.87 | 106 ± 42.6 | 1/39.1 |
| AKG129 | 7 | −7.84 ± 0.11 | 1 | −8.56 ± 0.04 | 7/1 | 0.83 ± 0.25 | 19.4 ± 7.7 | 1/23.4 |
| AKG141 | N.D. | N.D. | N.D. | N.D. | -/- | 4.4 ± 2.15 | 20.0 ± 1.8 | 1/10 |
| AKG142 | 8 | −7.67 ± 0.07 | 2 | −8.47 ± 0.05 | 4/1 | 5.71 ± 0.73 | 18.1 ± 1.52 | 1/3.2 |
| AKG143 | 2 | −8.35 ± 0.07 | 0.7 | −8.84 ± 0.08 | 2.9/1 | 3.2 ± 0.28 | 21.7 ± 9.1 | 1/6.8 |
| AKG144 | 0.5 | −8.94 ± 0.07 | 0.2 | −9.36 ± 0.03 | 2.5/1 | 5.39 ± 1.4 | 23.7 ± 5.9 | 1/4.2 |
| AKG-SK-145 | 2 | −8.43 ± 0.08 | 6 | −7.84 ± 0.05 | 1/3 | 7.5 ± 1.23 | 125.0 ± 13.8 | 1/16 |
| AKG-SK-146 | 0.6 | −8.94 ± 0.17 | 0.6 | −8.85 ± 0.06 | 1/1 | 6.37 ± 1.54 | 78.6 ± 9.8 | 1/15 |

N.D. means not determined,
n in the parenthesis indicates number of run

Structure-activity relationship (SAR) study was performed starting with the ligand AKG104 in which Asn was introduced as 5^{th} residue in place of Nle of TY045. It showed moderate binding affinities at all receptors (Kᵢ^{μ}=51 nM, Kᵢ^{δ}=17 nM, Kᵢ^{hNK1}=1.2 nM, Kᵢ^{rNK1}=20 nM, Table XI). Inversion of chirality at 5^{th} residue produced ligand AKG102, which displayed inferior biological profiles at all receptors (Kᵢ^{μ}=90 nM, Kᵢ^{δ}=54 nM, Kᵢ^{hNK1=2.3} nM, Kᵢ^{rNK1}=130 nM, Table XI). To examine the effect of length of side chain, Gln was introduced in place of Asn to get AKG105. It showed little better binding affinities at every receptor under study (Kᵢ^{μ}=38 nM, Kᵢ^{δ}=9 nM, Kᵢ^{hNK1}=1 nM, Kᵢ^{rNK1}=12 nM, Table XI). To be confirmed on the effect of inversion of chirality at 5^{th} residue, D-Gln containing AKG103 was designed and synthesized. This modification reduced the affinities for all the receptors (Kᵢ^{μ}=120 nM, Kᵢ^{δ}=72 nM, Kᵢ^{hNK1}=2.7 nM, Kᵢ^{rNK1}=110 nM, Table XI). As better results were observed with AKG105, further structural modifications were made.

N-methylation on 4^{th} residue i.e. Phe gave the ligand AKG129. This change made the resultant ligand more potent at opioid receptors while maintaining its affinity at NK1R (Kᵢ^{μ}=7 nM, Kᵢ^{δ}=1 nM, Kᵢ^{hNK1}=1 nM, Kᵢ^{rNK1}=19 nM, Table XI). AKG141, which was produced because of the replacement of Tyr at position from AKG129 by Dmt, became potent at opioid receptors (Kᵢ^{μ}=x nM, Kᵢ^{δ}=x nM, Kᵢ^{hNK1}=x nM, Kᵢ^{rNK1}=x nM, Table XI). AKG142, a ligand containing Phe(4-F) as 4^{th} residue instead of Phe as it was AKG105, became more potent (Kᵢ^{μ}=8 nM, Kᵢ^{δ}=2 nM, Kᵢ^{hNK1}=5.7 nM, Kᵢ^{rNK1}=18 nM, Table XI) compared to the parent ligand but furnished similar results when compared to those shown by AKG129. To examine the effect of N-methylation we introduced NMePhe(4-F) as 4^{th} residue, which produced the ligand AKG143. This modification further increased the potency at opioid receptors with no significance change of that at NK1R (Kᵢ^{μ}=2 nM, Kᵢ^{δ}=0.7 nM, Kᵢ^{hNK1}=3.2 nM, Kᵢ^{rNK1}=22 nM, Table XI). When Dmt was introduced, though the new ligand AKG144 became more potent at opioid receptors, and it showed small decrease at NK1R (Kᵢ^{μ}=0.5 nM, Kᵢ^{δ}=0.2 nM, Kᵢ^{hNK1}=5.4 nM, Kᵢ^{rNK1}=24 nM, Table XI). This reduction might be due to the interference of opioid pharmacophore in the affinity of NK1 pharmacophore. In an effort to reduce this interference we increased the length of the address region by introducing Gly as 6^{th} residue in ligand AKG-SK-145. But it further reduced affinities at rNK1 receptors (Kᵢ^{μ}=2 nM, Kᵢ^{δ}=6 nM, Kᵢ^{hNK1}=7.5 nM, Kᵢ^{rNK1}=120 nM, Table XI). Introduction of Dmt at 1^{st} position, which produced the ligand AKG-SK-146, provided improved potency at all receptors including rNK1 (Kᵢ^{μ}=0.6 nM, Kᵢ^{δ}=0.6 nM, Kᵢ^{hNK1}=6.4 nM, Kᵢ^{rNK1}=79 nM, Table XI).

Oligopeptides or ligands having a combination of dermorphin (H-Tyr-D-Ala-Phe-Gly-Tyr-Pro-Ser-NH₂), a naturally occurring and highly mu-selective ligand, and NK1 derived pharmacophores were also prepared and studies. Thus, structural features of dermorphin have been introduced into the multivalent ligands. N-Methylated unnatural amino acids also have been introduced during this study.

TABLE XII
Another Representative Oligopeptides of the Invention

| | |
|---|---|
| AKG114: | (SEQ ID NO: 32, where Tyr′ = Tyr, R on Ala, Gly and |
| | Leu is H, Phe′ = Phe, x = 0, and —XCH(R)—Ph(R′)₂ |
| | is —NHCH₂(3,5-difluorophenyl); |
| AKG118: | (same as AKG114 except Tyr′ at position 1 is Dmt); |
| AKG210: | (same as AKG114 except Phe′ = Phe(4-F)); |
| AKG211: | (same as AKG114 except R on Gly is methyl); |
| AKG212: | (same as AKG114 except Phe′ = Phe(4-F), |
| | R on Gly is methyl); |
| AKG213: | (same as AKG114 except Phe′ = NMePhe); |
| AKG214: | (same as AKG114 except x = 1 and AA = Ser); |
| AKG215: | (same as AKG114 except x = 2 and (AA)x = Pro-Ser). |

Here, we introduced the structural features of dermorphin (H-Tyr-D-Ala-Phe-Gly-Tyr-Pro-Ser-NH₂), a mu-selective peptide based opioid ligand. Compound AKG114 was designed by removing Ser from the C-terminal and connecting the remaining sequence of dermorphin with NK1 pharmacophore. In this ligand Pro was anticipated to influence binding affinities at opioid as well as NK1 receptors. It showed 5 times higher binding affinity for MOR compared to that for DOR and high affinity at NK1R (Kᵢ^{μ}=4 nM, Kᵢ^{δ}=19 nM, Kᵢ^{hNK1}=2 nM, Kᵢ^{rNK1}=40 nM, Table XII). Functional assay with this ligand displayed 4 times higher agonist activity at DOR compared to that at MOR (IC₅₀^{μ}=110 nM, IC₅₀^{δ}=29 nM, Kₑ^{NK1}=15 nM, Table XIII) Replacement of 1^{st} residue i.e. Tyr by Dmt resulted the molecule AKG118 and it exhibited higher binding affinity at opioid receptors while maintaining that NK1R (Kᵢ^{μ}=1 nM, Kᵢ^{δ}=3 nM, Kᵢ^{hNK1}=2.3 nM, Kᵢ^{rNK1}=25 nM, Table XII). The following oligopeptides were also synthesized AKG210, AKG211, AKG212, AKG213, AKG214, and AKG215 in an attempt to achieve higher selectivity for MOR over DOR.

TABLE XIII
Binding affinity results at opioid and NK1 receptors

| Ligand No. | Kᵢ^{u} (nM) | Log[IC₅₀±] | Kᵢ^{δ} (nM) | Log[IC₅₀±] | Kᵢ^{u}/Kᵢ^{δ} | Kᵢ^{hNK1} (nm) | Kᵢ^{rNK1} (nm) | Kᵢ^{hNK1}/Kᵢ^{rNK1} |
|---|---|---|---|---|---|---|---|---|
| AKG114 | 4 | −8.02 ± 0.04 | 19 | −7.40 ± 0.07 | 1/5 | 1.96 ± 0.37 | 39.6 ± 0.41 | 1/22.2 |
| | (n = 2) | | (n = 2) | | | (n = 6) | (n = 6) | |
| QXP04 | 10 | −7.66 ± 0.05 | 69 | −6.82 ± 0.06 | 1/7 | 3.8 ± 0.54 | 13.0 ± 1.7 | 1/3.4 |
| | (n = 2) | | (n = 2) | | | (n = 6) | (n = 6) | |
| AKG118 | 1 | −8.65 ± 0.12 | 3 | −8.20 ± 0.04 | 1/3 | 2.34 ± 0.39 | 25.4 ± 4.17 | 1/11 |
| | (n = 2) | | (n = 2) | | | (n = 6) | (n = 6) | |
| AKG210 | N.D. | N.D. | N.D. | N.D. | -/- | 385 ± 2.82 | | -/- |
| | | | | | | (n = 6) | | |

N.D. means not determined,
n in the parenthesis indicates number of run

TABLE XIV
Functional assay results

| Compd. | GPI (MOR) | MVD (DOR) | GPI/MVD | GPI/LMMP (NK1R) | |
|---|---|---|---|---|---|
| Number | IC₅₀ (nM) | IC₅₀ (nM) | IC₅₀ ratio | Agonist | Kₑ (nM) ± S.E.M. |
| AKG114 | 111.5 ± 12.8 | 28.98 ± 6.70 | 4/1 | None at 300 nM | 15.0 ± 4.2 |
| QXP04 | 389.2 ± 179.7 | 16.59 ± 4.57 | 23/1 | None at 1 uM | 36.3 ± 16.2 |

For every sample, the number of run was six at each receptor

In one embodiment, compounds having a combination of morphiceptin (H-Tyr-Pro-Phe-Pro-NH₂), a synthetic and highly mu-selective ligand, and NK1 derived pharmacophores are provided. For the first time, structural features of morphiceptin have been incorporated into the multivalent ligands.

TABLE XV

| | |
|---|---|
| AKG196: | (SEQ ID NO: 43 where Tyr′ = Tyr, Z is absent, |
| | Phe′ = Phe, x = 0, R on Leu and Trp is H |
| | and —XCH(R)—Ph(R′)₂ is —NHCH₂(3,5-difluorophenyl); |
| QXP08: | (same as AKG196 except R on Trp is methyl); |
| AKG197: | (same as AKG196 except x = 1 and AA = Pro); |
| AKG198: | (same as AKG196 except x = 2, (AA)x = |
| | Gly-Nle, and R on Trp is methyl); |
| AKG200: | (same as AKG196 except x = 1 and AA = Gly); |
| AKG201: | (same as AKG196 except x = 1 and AA = NMeGly); |
| AKG202: | (SEQ ID NO: 33, where Tyr′ = Tyr, R on Gly and |
| | Leu is H, Phe′ = Phe, x = 0, and |
| | —XCH(R)—Ph(R′)₂ is —NHCH₂(3,5-difluorophenyl); |
| AKG203: | (same as AKG202 except Phe′ = NMePhe). |

Oligopeptides based on the structural feature of mu-selective pharmacophore morphiceptin including, but not limited to, AKG196, AKG197, AKG198, AKG200, AKG201, AKG202 and AKG203 have been synthesized and characterized (Table XV).

Taking into consideration of structural features of endogenous opioid peptides endomorphin-1 (H-Tyr-Pro-Trp-Phe-NH₂) and endomorphin-2 (H-Tyr-Pro-Phe-Phe-NH₂), additional oligopeptides were prepared.

TABLE XVI

| | |
|---|---|
| AKG221: | H-Tyr-Pro-Trp-Phe-Pro-Leu-Trp-NH-Bn |
| | (3′,5′-(CF₃)₂) (SEQ ID NO: 44); |
| AKG222: | H-Tyr-Pro-Phe-Phe-Pro-Leu-Trp-NH-Bn |
| | (3′,5′-(CF₃)₂) (SEQ ID NO: 45); |
| AKG223: | H-Tyr-Pro-Trp-NMePhe-Pro-Leu-Trp-NH- |
| | Bn(3′,5′-(CF₃)₂) (SEQ ID NO: 46); |
| AKG224: | H-Tyr-Pro-Phe-NMePhe-Pro-Leu-Trp-NH- |
| | Bn(3′,5′-(CF₃)₂) (SEQ ID NO: 47); |
| AKG225: | H-Tyr-Pro-NMeTrp-NMePhe-Pro-Leu-Trp- |
| | NH-Bn(3′,5′-(CF₃)₂) (SEQ ID NO: 48); |
| AKG226: | H-Tyr-Pro-NMePhe-NMePhe-Pro-Leu-Trp- |
| | NH-Bn(3′,5′-(CF₃)₂) (SEQ ID NO: 49); |
| AKG227: | (SEQ ID NO: 43 where Tyr′ = Tyr, |
| | Z = Trp, Phe′ = NMePhe, x = 1, |
| | AA = 4-Abz, R on Leu and Trp is H |
| | and -XCH(R)-Ph(R′)₂ is -NHCH2(3,5- |
| | difluorophenyl); |
| AKG228: | (same as AKG227 except AA = 4-Amb); |
| AKG229: | (same as AKG227 except AA = 4-Apac); |
| AKG230: | (same as AKG227 except AA = 4-Ampa). |

In particular, multivalent ligands based on the structural feature of mu-selective pharmacophore endomorphins were designed and synthesized including, but not limited to, AKG221, AKG222, AKG223, AKG224, AKG225, AKG226, AKG227, AKG228, AKG229, and AKG230 (Table XVI). Binding and affinity results of these oligopeptides are shown in Table XVII.

TABLE XVII
Binding affinity results at opioid and NK1 receptors

| Ligand No. | Kᵢ^{u} (nM) | Log[IC₅₀±] | Kᵢ^{δ} (nM) | Log[IC₅₀±] | Kᵢ^{u}/Kᵢ^{δ} | Kᵢ^{hNK1} (nm) | Kᵢ^{rNK1} (nm) | Kᵢ^{hNK1}/Kᵢ^{rNK1} |
|---|---|---|---|---|---|---|---|---|
| AKG221 | N.D. | N.D. | N.D. | N.D. | -/- | 3.34 ± 0.32 | 38.46 ± 3.6 | 1/11.5 |
| | | | | | | (n = 6) | (n = 6) | |
| AKG222 | N.D. | N.D. | N.D. | N.D. | -/- | 4.19 ± 0.82 | 32.4 ± 9.0 | 1/7.7 |
| | | | | | | (n = 6) | (n = 6) | |
| AKG223 | N.D. | N.D. | N.D. | N.D. | -/- | 3.59 ± 0.3 | 10.11 ± 6.3 | 1/2.8 |
| | | | | | | (n = 6) | (n = 6) | |
| AKG224 | N.D. | N.D. | N.D. | N.D. | -/- | 3.39 ± 0.85 | 33.74 ± 2.78 | 1/10 |
| | | | | | | (n = 6) | (n = 6) | |
| AKG225 | N.D. | N.D. | N.D. | N.D. | -/- | 14.8 ± 1.4 | 301.9 ± 78.5 | 1/20 |
| | | | | | | (n = 6) | (n = 6) | |
| AKG226 | N.D. | N.D. | N.D. | N.D. | -/- | 5.90 ± 0.5 | 31.72 ± 13.3 | 1/5.4 |
| | | | | | | (n = 6) | (n = 6) | |

N.D. means not determined,
n in the parenthesis indicates number of run

### In Vitro Metabolic Stability

To check the stability of some of the ligands, metabolic stability study was conducted by incubating the ligands in rat plasma at 37° C. Ligand AKG115 (T_{1/2}: >24 h) and AKG127 (T_{1/2}: >24 h) showed significant enhancement in stability compared to both TY027 (T_{1/2}: 4.8 h) and TY032 (T_{1/2}: >6 h). Compound AKG190 was also tested for its metabolic stability to know the effect of 4^{th} residue. It showed lower half-life (T_{1/2}: <2 h) compared to that for AKG115 and AKG127. These results suggest that presence of Dmt at 1^{st} position is playing a role in enhancing the metabolic stability.

### In Vivo Results

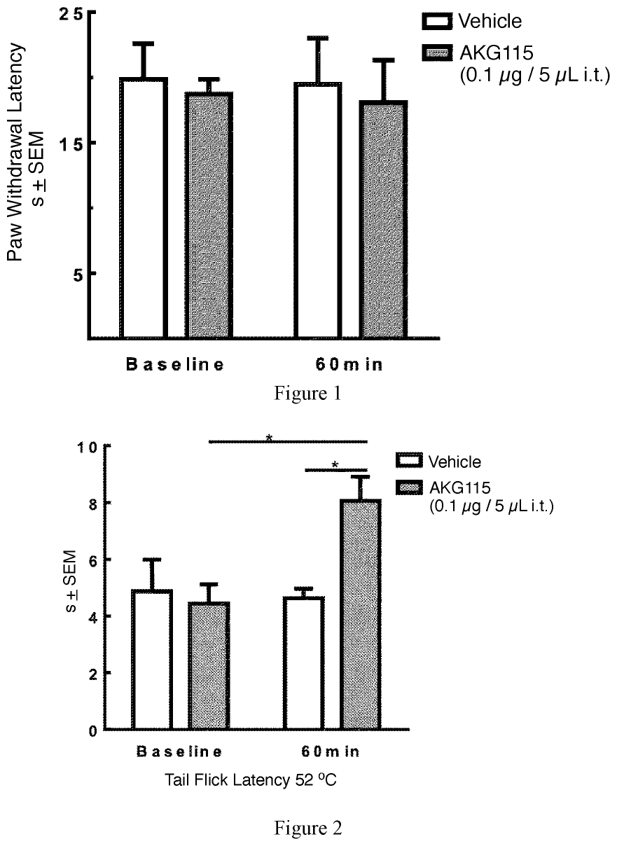

Comparison of in vitro results suggest that number of compounds including AKG115, AKG116, AKG127, AKG113, AKG-CRA-177, AKG114 and AKG118 may have antinociceptive activity in vivo. Compounds AKG115 and AKG127 were chosen for preliminary in vivo studies. The efficacy of spinal AKG115 (0.1 μg in 5 μL) or vehicle were evaluated in rats using a radiant heat assay to elicit a paw withdrawal reflex. Paw withdrawal latencies (PWLs) of rats given AKG115 were not significantly higher than vehicle-treated rats and baseline values 60 min after the injection (FIG. 1). The dose was increased to 10 μg in 5 μl; however, motor skills were impaired rendering analysis of PWLs inconclusive (data not shown). The structural modification made to compound AKG115 to create compound AKG127 indicated that in in vivo activity may be more pronounced in the latter. Studies in a mouse model of acute thermal pain showed that tail flick latencies (TFLs) of mice administered AKG127 (0.1 μg in 5 μl, i.t.) were significantly higher than vehicle-treated mice and baseline values 60 min after injection (p=0.04 compared to vehicle treatment group, p=0.02 compared to baseline value; FIG. 2).

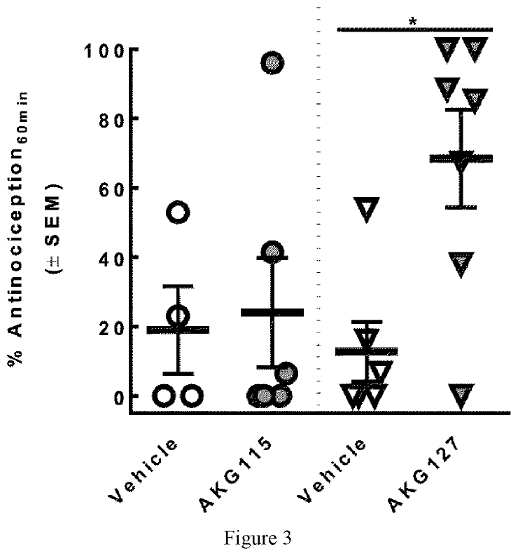

To determine if the structural modifications significantly impacted the maximal percent activity of AKG115 and AKG127, we calculated the % antinociception (Equation 1) at the same dose is shown in FIG. 3.
For both studies, maximal percent efficacy was calculated and expressed as:

$$\% Antinociception = 100 * \frac{test latency after drug treatment - baseline latency}{cutoff - baseline latency} \qquad Equation 1$$

These studies show in vivo activity of ligands AKG115 and AKG127 in a model of acute thermal pain in two species. Despite having high binding affinity and in vitro functional activity, the maximal level of antinociception observed after AKG115 administration was minimal; in contrast, AKG127 administration was approximately 70%. These data suggest that structural modifications in the linker region of the opioid agonist/NK1 antagonist enhanced in vivo activity.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. An oligopeptide of the formula:
AA¹-Q-Pro-AA²-AA³
where
AA¹ is Tyr or Dmt;
AA² is Leu or methylate Leu;
AA³ is Trp or methylated Trp each of which is covalently linked to optionally substituted benzyl amine or (optionally substituted phenyl)ethan-1-amine; and
Q is a moiety of the formula:
-(D)-NRAla-Phe′-NRGly-Tyr′-Pro-Ser-, or
-Pro-[Z]_{b}-Phe′-Pro-;
-AA⁴-AA⁵-AA⁶-AA⁷-Pro-Ser- (i)
AA⁴ is (D)-Ala or methylated (D)-Ala;
AA⁵ is Phe, Phe(4-F), methylated Phe, or methylated Phe(4-F);
AA⁶ is Gly or methylated Gly; and
AA⁷ is Tyr or Dmt; or
-Pro-(AA⁸)ₐ-AA⁹-Pro- (ii)
a is 0 or 1;
AA⁸ is Phe or Trp; and
AA⁹ is Phe, Phe(4-F), methylated Phe, or methylated Phe(4-F), provided at least one of AA², AA³, AA⁵, or AA⁹ is methylated.

2. The oligopeptide of claim 1, wherein AA³ is covalently linked to benzyl amine, 3,5-di(trifluoromethyl)benzylamine, 1-phenylethan-1-amine, or 1-(3,5-di(trifluoromethyl)phenyl)ethan-1-amine.

3. The oligopeptide of claim 2, wherein AA3 is covalently linked to 3,5-di(trifluoromethyl)benzylamine or 1-(3,5-di(trifluoromethyl)phenyl)ethan-1-amine.

4. The oligopeptide of claim 1, wherein Q is a moiety of the formula:
-AA⁴-AA⁵-AA⁶-AA⁷-Pro-Ser-
wherein AA⁴, AA⁵, AA⁶, and AA⁷ are as defined in claim 1.

5. The oligopeptide of claim 1, wherein Q is a moiety of the formula:
-Pro-(AA⁸)ₐ-AA⁹-Pro-
wherein a, AA⁸, and AA⁹ are as defined in claim 1.

6. A method for treating pain comprising administering a subject in need of such a treatment a therapeutically effective amount of a compound of an oligopeptide of the formula:
AA¹-Q-Pro-AA²-AA³
where
AA¹ is Tyr or Dmt;
AA² is Leu or methylate Leu;
AA³ is Trp or methylated Trp each of which is covalently linked to optionally substituted benzyl amine or (optionally substituted phenyl)ethan-1-amine; and
Q is a moiety of the formula:
-(D)-NRAla-Phe′-NRGly-Tyr′-Pro-Ser-, or
-Pro-[Z]_{b}-Phe′-Pro-;
-AA⁴-AA⁵-AA⁶-AA⁷-Pro-Ser- (i)
AA⁴ is (D)-Ala or methylated (D)-Ala;
AA⁵ is Phe, Phe(4-F), methylated Phe, or methylated Phe(4-F);
AA⁶ is Gly or methylated Gly; and
AA⁷ is Tyr or Dmt; or
-Pro-(AA⁸)ₐ-AA⁹-Pro- (ii)
a is 0 or 1;
AA⁸ is Phe or Trp; and
AA⁹ is Phe, Phe(4-F), methylated Phe, or methylated Phe(4-F), provided at least one of AA², AA³, AA⁵, or AA⁹ is methylated.

7. The method of claim 6, wherein pain is an acute pain.

8. The method of claim 6, wherein pain is a chronic pain.

9. The method of claim 6, wherein AA³ is covalently linked to benzyl amine, 3,5-di(trifluoromethyl)benzylamine, 1-phenylethan-1-amine, or 1-(3,5-di(trifluoromethyl)phenyl)ethan-1-amine.

10. The method of claim 9, wherein AA³ is covalently linked to 3,5-di(trifluoromethyl)benzylamine or 1-(3,5-di(trifluoromethyl)phenyl)ethan-1-amine.

11. The method of claim 6, wherein Q is a moiety of the formula:
-AA⁴-AA⁵-AA⁶-AA⁷-Pro-Ser-
wherein AA⁴, AA⁵, AA⁶, and AA⁷ are as defined in claim 6.

12. The method of claim 6, wherein Q is a moiety of the formula:
Pro(AA⁸)ₐAA⁹Pro-
wherein a, AA⁸, and AA⁹ are as defined in claim 6.

13. A pharmaceutical composition comprising a pharmaceutically acceptable excipient and an oligopeptide of the formula:
AA¹-Q-Pro-AA²-AA³
where
AA¹ is Tyr or Dmt;
AA² is Leu or methylate Leu;
AA³ is Trp or methylated Trp each of which is covalently linked to optionally substituted benzyl amine or (optionally substituted phenyl)ethan-1-amine; and
Q is a moiety of the formula:
-(D)-NRAla-Phe′-NRGly-Tyr′-Pro-Ser-, or
-Pro-[Z]_{b}-Phe′-Pro-;
-AA⁴-AA⁵-AA⁶-AA⁷-Pro-Ser- (i)
AA⁴ is (D)-Ala or methylated (D)-Ala;
AA⁵ is Phe, Phe(4-F), methylated Phe, or methylated Phe(4-F);
AA⁶ is Gly or methylated Gly; and
AA⁷ is Tyr or Dmt; or
-Pro-(AA⁸)ₐ-AA⁹-Pro- (ii)
a is 0 or 1;
AA⁸ is Phe or Trp; and
AA⁹ is Phe, Phe(4-F), methylated Phe, or methylated Phe(4-F), provided at least one of AA², AA³, AA⁵, or AA⁹ is methylated.


SEQUENCE LISTING (62 sequences)

SEQ ID NO: 1 - protein, 5 aa, Artificial Sequence
  Opiod Agonist
YGGFM

SEQ ID NO: 2 - protein, 5 aa, Artificial Sequence
  Opioid Agonist
TGGFL

SEQ ID NO: 3 - protein, 5 aa, Artificial Sequence
  Opioid Agonist
  MISC_FEATURE (2)..(2): D-Isomer
  MISC_FEATURE (4)..(4): Amino group is methylated.
  MISC_FEATURE (5)..(5): Carboxylic Acid group (-CO2H) is reduced to hydroxyl group (-OH)
TAGFG

SEQ ID NO: 4 - protein, 7 aa, Artificial Sequence
  Opioid Agonist
  MISC_FEATURE (2)..(2): D-Isomer
  MISC_FEATURE (7)..(7): Carboxylic Acid (-CO2H) is replaced with Amide group (-CONH2)
TAFGTPS

SEQ ID NO: 5 - protein, 4 aa, Artificial Sequence
  Opioid Agonist
  MISC_FEATURE (4)..(4): Carboxylic acid group is replaced with an amide group
TPFP

SEQ ID NO: 6 - protein, 4 aa, Artificial Sequence
  Opioid Agonist
  MISC_FEATURE (4)..(4): Carboxylic acid is replaced with an amide group
TPWF

SEQ ID NO: 7 - protein, 4 aa, Artificial Sequence
  Opioid Agonist
  MISC_FEATURE (4)..(4): Carboxylic acid group is replaced with an amide group.
TPFF

SEQ ID NO: 8 - protein, 8 aa, Artificial Sequence
  NK1 antagonist
  MISC_FEATURE (2)..(2): D-Isomer
  MISC_FEATURE (8)..(8): Carboxylic acid (-CO2H) is replaced with an amide group (-CONHR, where R is substituted with 3′,5′-ditrifluoromethylbenzyl)
TAGFMPLW

SEQ ID NO: 9 - protein, 8 aa, Artificial Sequence
  TY005
  MISC_FEATURE (2)..(2): D-isomer
  MISC_FEATURE (8)..(8): Carboxylic acid (-CO2H) is replaced with -COONHR, where R is 3′,5′-ditrifluoromethylbenzyl
TAGFMPLW

SEQ ID NO: 10 - protein, 7 aa, Artificial Sequence
  Peptide #10
  MISC_FEATURE (1)..(1): Tyr and its derivatives, e.g., Dmt etc.
  MISC_FEATURE (2)..(2): D-isomer
  MISC_FEATURE (2)..(2): alpha-amino group is optionally substituted with an alkyl such as methyl
  MISC_FEATURE (3)..(3): alpha-amino group is optionally substituted with an alkyl such as methyl
  MISC_FEATURE (4)..(4): Phe and its derivatives, e.g., NMePhe, Phe(4-F), etc.
  MISC_FEATURE (6)..(6): alpha-amino group is optionally substituted with an alkyl such as methyl
  MISC_FEATURE (7)..(7): alpha-amino group is optionally substituted with an alkyl such as methyl
  MISC_FEATURE (7)..(7): Carboxy terminal group (-CO2H) is replaced with -CONR, where R is benzyl which is optionally substituted in 3′-, and 5′- position of the phenyl ring with H, CH3, CF3, etc. and the methylene group of the benzyl is optionally substituted with H, CH3, etc.
XAGXPLW

SEQ ID NO: 11 - protein, 7 aa, Artificial Sequence
  Compound of the invention
  MISC_FEATURE (2)..(2): D-isomer
  MISC_FEATURE (4)..(4): alpha-amino group is substituted with methyl
  MISC_FEATURE (7)..(7): Carboxy group (-CO2H) is replaced with -CONHR, where R is 3′,5′-ditrifluoromethylbenzyl
YAGFPLW

SEQ ID NO: 12 - protein, 7 aa, Artificial Sequence
  Compound 12
  MISC_FEATURE (1)..(1): Dmt
  MISC_FEATURE (2)..(2): D-isomer
  MISC_FEATURE (4)..(4): alpha-amino group is substituted with methyl
  MISC_FEATURE (7)..(7): Carboxy group (-CO2H) is replaced with -CONHR, where R is 3′,5′-ditrifluoromethylbenzyl
XAGFPLW

SEQ ID NO: 13 - protein, 7 aa, Artificial Sequence
  Compound 13
  MISC_FEATURE (1)..(1): Dmt
  MISC_FEATURE (2)..(2): D-isomer
  MISC_FEATURE (7)..(7): Carboxy group (-CO2H) is replaced with -CONHR, where R is 3′,5′-ditrifluoromethylbenzyl
XAGFPLW

SEQ ID NO: 14 - protein, 7 aa, Artificial Sequence
  Compound 14
  MISC_FEATURE (1)..(1): Dmt
  MISC_FEATURE (2)..(2): D-isomer
  MISC_FEATURE (4)..(4): 4-position of the phenyl group of the phenylalanine is substituted with fluoride
  MISC_FEATURE (7)..(7): Carboxy group (-CO2H) is replaced with -CONHR, where R is 3′,5′-ditrifluoromethylbenzyl
XAGFPLW

SEQ ID NO: 15 - protein, 7 aa, Artificial Sequence
  Compound 15
  MISC_FEATURE (1)..(1): Dmt
  MISC_FEATURE (2)..(2): D-isomer
  MISC_FEATURE (4)..(4): 4-position of the phenyl group of the phenylalanine is substituted with fluoride
  MISC_FEATURE (4)..(4): alpha-amino group is substituted with methyl
  MISC_FEATURE (7)..(7): Carboxy group (-CO2H) is replaced with -CONHR, where R is 3′,5′-ditrifluoromethylbenzyl
XAGFPLW

SEQ ID NO: 16 - protein, 7 aa, Artificial Sequence
  Compound 16
  MISC_FEATURE (1)..(1): Dmt
  MISC_FEATURE (2)..(2): D-isomer
  MISC_FEATURE (4)..(4): 4-position of the phenyl group of the phenylalanine is substituted with chloride
  MISC_FEATURE (7)..(7): Carboxy group (-CO2H) is replaced with -CONHR, where R is 3′,5′-ditrifluoromethylbenzyl
XAGFPLW

SEQ ID NO: 17 - protein, 7 aa, Artificial Sequence
  Compound 17
  MISC_FEATURE (1)..(1): Dmt
  MISC_FEATURE (2)..(2): D-isomer
  MISC_FEATURE (4)..(4): 4-position of the phenyl group of the phenylalanine is substituted with bromide
  MISC_FEATURE (7)..(7): Carboxy group (-CO2H) is replaced with -CONHR, where R is 3′,5′-ditrifluoromethylbenzyl
XAGFPLW

SEQ ID NO: 18 - protein, 7 aa, Artificial Sequence
  Compound 18
  MISC_FEATURE (1)..(1): Dmt
  MISC_FEATURE (2)..(2): D-isomer
  MISC_FEATURE (4)..(4): 4-position of the phenyl group of the phenylalanine is substituted with iodide
  MISC_FEATURE (7)..(7): Carboxy group (-CO2H) is replaced with -CONHR, where R is 3′,5′-ditrifluoromethylbenzyl
XAGFPLW

SEQ ID NO: 19 - protein, 10 aa, Artificial Sequence
  Compound 19 (General Structure)
  MISC_FEATURE (1)..(1): Tyr and its derivatives, e.g., Dmt etc.
  MISC_FEATURE (2)..(2): D-isomer
  MISC_FEATURE (2)..(2): alpha-amino group is optionally substituted with alkyl, such as methyl, etc.
  MISC_FEATURE (3)..(3): alpha-amino group is optionally substituted with alkyl, such as methyl, etc.
  MISC_FEATURE (4)..(4): Phe and its derivatives, e.g., NMePhe, Phe(4-F) etc.
  MISC_FEATURE (5)..(7): each amino acid is independently natual/unnatural amino acid, e.g., Nle, Gly, beta-Ala, 4-Abu, Ahx, 4-Amb, 4-Abz, 4-Apac, 4-Ampa etc.
  MISC_FEATURE (9)..(9): alpha-amino group is optionally substituted with alkyl, such as methyl, etc.
  MISC_FEATURE (10)..(10): alpha-amino group is optionally substituted with alkyl, such as methyl, etc.
  MISC_FEATURE (10)..(10): Carboxy group (-CO2H) is replaced with -CONHR, where R is 3′,5′-ditrifluoromethylbenzyl
  MISC_FEATURE (10)..(10): Carboxy terminal group (-CO2H) is replaced with -CONR, where R is benzyl which is optionally substituted in 3′-, and 5′- position of the phenyl ring with H, CH3, CF3, etc. and the methylene group of the benzyl is optionally substituted with H, CH3, etc.
XAGXXXXPLW

SEQ ID NO: 20 - protein, 8 aa, Artificial Sequence
  Compound 20
  MISC_FEATURE (2)..(2): D-isomer
  MISC_FEATURE (4)..(4): alpha-amino group is substituted with methyl
  MISC_FEATURE (5)..(5): Nle
  MISC_FEATURE (8)..(8): Carboxy group (-CO2H) is replaced with -CONHR, where R is 3′,5′-ditrifluoromethylbenzyl
YAGFXPLW

SEQ ID NO: 21 - protein, 8 aa, Artificial Sequence
  Compound 21
  MISC_FEATURE (2)..(2): D-isomer
  MISC_FEATURE (4)..(4): alpha-amino group is substituted with methyl
  MISC_FEATURE (8)..(8): Carboxy group (-CO2H) is replaced with -CONHR, where R is 3′,5′-ditrifluoromethylbenzyl
YAGFGPLW

SEQ ID NO: 22 - protein, 8 aa, Artificial Sequence
  Compound 22
  MISC_FEATURE (2)..(2): D-isomer
  MISC_FEATURE (4)..(4): 4-position of the phenyl group of the phenylalanine is substituted with fluoride
  MISC_FEATURE (8)..(8): Carboxy group (-CO2H) is replaced with -CONHR, where R is 3′,5′-ditrifluoromethylbenzyl
YAGFGPLW

SEQ ID NO: 23 - protein, 8 aa, Artificial Sequence
  Compound 23
  MISC_FEATURE (2)..(2): D-isomer
  MISC_FEATURE (4)..(4): alpha-amino group is substituted with methyl
  MISC_FEATURE (4)..(4): 4-position of the phenyl group of the phenylalanine is substituted with fluoride
  MISC_FEATURE (8)..(8): Carboxy group (-CO2H) is replaced with -CONHR, where R is 3′,5′-ditrifluoromethylbenzyl
YAGFGPLW

SEQ ID NO: 24 - protein, 8 aa, Artificial Sequence
  Compound 24
  MISC_FEATURE (2)..(2): D-isomer
  MISC_FEATURE (4)..(4): alpha-amino group is substituted with methyl
  MISC_FEATURE (5)..(5): beta-alanine
  MISC_FEATURE (8)..(8): Carboxy group (-CO2H) is replaced with -CONHR, where R is 3′,5′-ditrifluoromethylbenzyl
YAGFXPLW

SEQ ID NO: 25 - protein, 8 aa, Artificial Sequence
  Compound 25
  MISC_FEATURE (2)..(2): D-Isomer
  MISC_FEATURE (4)..(4): alpha-amino group is substituted with methyl
  MISC_FEATURE (5)..(5): gamma-Abu
  MISC_FEATURE (8)..(8): gamma-AbuCarboxy group (-CO2H) is replaced with -CONHR, where R is 3′,5′-ditrifluoromethylbenzyl
YAGFXPLW

SEQ ID NO: 26 - protein, 8 aa, Artificial Sequence
  Compound 26
  MISC_FEATURE (2)..(2): D-Isomer
  MISC_FEATURE (4)..(4): alpha-amino group is substituted with methyl
  MISC_FEATURE (5)..(5): 6-Ahx
  MISC_FEATURE (8)..(8): Carboxy group (-CO2H) is replaced with -CONHR, where R is 3′,5′-ditrifluoromethylbenzyl
YAGFXPLW

SEQ ID NO: 27 - protein, 8 aa, Artificial Sequence
  Compound 27
  MISC_FEATURE (2)..(2): D-isomer
  MISC_FEATURE (4)..(4): alpha-amino group is substituted with methyl
  MISC_FEATURE (5)..(5): 4-Amb
  MISC_FEATURE (8)..(8): Carboxy group (-CO2H) is replaced with -CONHR, where R is 3′,5′-ditrifluoromethylbenzyl
YAGFXPLW

SEQ ID NO: 28 - protein, 8 aa, Artificial Sequence
  Compound 28
  MISC_FEATURE (2)..(2): D-isomer
  MISC_FEATURE (4)..(4): alpha-amino group is substituted with methyl
  MISC_FEATURE (5)..(5): 4-Abz
  MISC_FEATURE (8)..(8): Carboxy group (-CO2H) is replaced with -CONHR, where R is 3′,5′-ditrifluoromethylbenzyl
YAGFXPLW

SEQ ID NO: 29 - protein, 8 aa, Artificial Sequence
  Compound 29
  MISC_FEATURE (2)..(2): D-isomer
  MISC_FEATURE (4)..(4): alpha-amino group is substituted with methyl
  MISC_FEATURE (5)..(5): 4-Apac
  MISC_FEATURE (8)..(8): 4-ApacCarboxy group (-CO2H) is replaced with -CONHR, where R is 3′,5′-ditrifluoromethylbenzyl
YAGFXPLW

SEQ ID NO: 30 - protein, 8 aa, Artificial Sequence
  Compound 30
  MISC_FEATURE (2)..(2): D-isomer
  MISC_FEATURE (4)..(4): alpha-amino group is substituted with methyl
  MISC_FEATURE (5)..(5): 4-Ampa
  MISC_FEATURE (8)..(8): 4-AmpaCarboxy group (-CO2H) is replaced with -CONHR, where R is 3′,5′-ditrifluoromethylbenzyl
YAGFXPLW

SEQ ID NO: 31 - protein, 10 aa, Artificial Sequence
  General Structure 31
  MISC_FEATURE (1)..(1): Tyr and its derivatives e.g., Dmt
  MISC_FEATURE (2)..(2): D-isomer
  MISC_FEATURE (3)..(3): alpha-amino group is optionally substituted with methyl
  MISC_FEATURE (4)..(4): 4-position of the phenyl group of the phenylalanine is optionally substituted with fluoride and alpha-amino group is optionally subsittued with methyl
  MISC_FEATURE (5)..(7): can be absent provided at least one is present and each is indenependently Ser, D-Ser, Homo-Ser, Lys, Orn, Dab, Dap, Ser-4-Apac, Asn, D-Asn, Gln, D-Gln, Gln-4-Apac
  MISC_FEATURE (9)..(9): alpha-amino group is optionally substituted with methyl
  MISC_FEATURE (10)..(10): alpha-amino group is optionally substituted with methyl
  MISC_FEATURE (10)..(10): Carboxy terminal group (-CO2H) is replaced with -CONR, where R is benzyl which is optionally substituted in 3′-, and 5′- position of the phenyl ring with H, CH3, CF3, etc. and the methylene group of the benzyl is optionally substituted with CH3, etc.
XAGXXXXPLW

SEQ ID NO: 32 - protein, 11 aa, Artificial Sequence
  Generic Structure 32
  MISC_FEATURE (1)..(1): Tyr and its derivatives, e.g., Dmt etc.
  MISC_FEATURE (2)..(2): D-isomer and alpha-amino group is optionally substituted with methyl etc.
  MISC_FEATURE (3)..(3): Phe and its derivatives, e.g., NMePhe, Phe(4-F), etc.
  MISC_FEATURE (4)..(4): alpha-amino group is optionally substituted with methyl etc.
  MISC_FEATURE (5)..(5): Tyr and its derivatives, e.g., Dmt etc.
  MISC_FEATURE (6)..(8): each is independently (1) can be absent or (2) is indpendently natural/unnatural amino acid, e.g., 4-Amb, 4-Apac, Lys, etc.; X=NH, NMe, etc.
  MISC_FEATURE (10)..(10): alpha-amino group is optionally substituted with methyl, etc.
  MISC_FEATURE (11)..(11): Carboxy terminal group (-CO2H) is replaced with -CONR, where R is benzyl which is optionally substituted in 3′-, and 5′- position of the phenyl ring with H, CH3, CF3, etc. and the methylene group of the benzyl is optionally substituted with CH3, etc.
XAXGXXXXPLW

SEQ ID NO: 33 - protein, 10 aa, Artificial Sequence
  Generic Structure 33
  MISC_FEATURE (1)..(1): Tyr and its derivatives e.g., Dmt etc.
  MISC_FEATURE (3)..(3): R is H, Me, etc.
  MISC_FEATURE (4)..(4): Phe and its derivatives, e.g., NMePhe, Phe(4-F) etc.
  MISC_FEATURE (5)..(7): each is independently natural/unnatural amino acid e.g., AA[[=AA]]=4-Amb, 4-Apac, Lys, etc.
  MISC_FEATURE (8)..(8): R is H, Me etc.
  MISC_FEATURE (9)..(9): R is H, Me etc.
  MISC_FEATURE (10)..(10): Carboxy terminal group (-CO2H) is replaced with -CONR, where R is benzyl which is optionally substituted in 3′-, and 5′- position of the phenyl ring with H, CH3, CF3, etc. and the methylene group of the benzyl is optionally substituted with H, CH3, etc.
XPGXXXXPLW

SEQ ID NO: 34 - protein, 6 aa, Artificial Sequence
  Compound 34
  MISC_FEATURE (6)..(6): Carboxy group (-CO2H) is replaced with -CONHR, where R is 3′,5′-ditrifluoromethylbenzyl
TPFPLW

SEQ ID NO: 35 - protein, 7 aa, Artificial Sequence
  Compound 35
  MISC_FEATURE (7)..(7): Carboxy group (-CO2H) is replaced with -CONHR, where R is 3′,5′-ditrifluoromethylbenzyl
YPFPPLW

SEQ ID NO: 36 - protein, 8 aa, Artificial Sequence
  Compound 36
  MISC_FEATURE (5)..(5): Nle
  MISC_FEATURE (8)..(8): Carboxy group (-CO2H) is replaced with -CONHR, where R is 3′,5′-ditrifluoromethylbenzyl
YPFGXPLW

SEQ ID NO: 37 - protein, 8 aa, Artificial Sequence
  Compound 37
  MISC_FEATURE (5)..(5): 4-Amb
  MISC_FEATURE (8)..(8): Carboxy group (-CO2H) is replaced with -CONHR, where R is 3′,5′-ditrifluoromethylbenzyl
YPFPXPLW

SEQ ID NO: 38 - protein, 8 aa, Artificial Sequence
  Compound 38
  MISC_FEATURE (5)..(5): 4-Amb
  MISC_FEATURE (8)..(8): Carboxy group (-CO2H) is replaced with -CONHR, where R is 3′,5′-ditrifluoromethylbenzyl
YPFPXPLW

SEQ ID NO: 39 - protein, 7 aa, Artificial Sequence
  Compound 39
  MISC_FEATURE (7)..(7): Carboxy group (-CO2H) is replaced with -CONHR, where R is 3′,5′-ditrifluoromethylbenzyl
YPFGPLW

SEQ ID NO: 40 - protein, 7 aa, Artificial Sequence
  Compound 40
  MISC_FEATURE (4)..(4): alpha-amino group is substituted with methyl
  MISC_FEATURE (7)..(7): Carboxy group (-CO2H) is replaced with -CONHR, where R is 3′,5′-ditrifluoromethylbenzyl
YPFGPLW

SEQ ID NO: 41 - protein, 7 aa, Artificial Sequence
  Compound 41
  MISC_FEATURE (7)..(7): Carboxy group (-CO2H) is replaced with -CONHR, where R is 3′,5′-ditrifluoromethylbenzyl
YPGFPLW

SEQ ID NO: 42 - protein, 7 aa, Artificial Sequence
  Compound 42
  MISC_FEATURE (4)..(4): alpha-amino group is substituted with methyl
  MISC_FEATURE (7)..(7): Carboxy group (-CO2H) is replaced with -CONHR, where R is 3′,5′-ditrifluoromethylbenzyl
YPGFPLW

SEQ ID NO: 43 - protein, 10 aa, Artificial Sequence
  Generic Structure 43
  MISC_FEATURE (1)..(1): Tyr or its derivative e.g., Dmt etc.
  MISC_FEATURE (3)..(3): Phe or its derivative, e.g., NMePhe, Phe(4-F) etc. or Trp or its derivative, e.g., NMeTrp etc.
  MISC_FEATURE (4)..(4): Phe or its derivative, e.g., NMePhe, Phe(4-F) etc.
  MISC_FEATURE (5)..(7): each is independently natural/unnatural amino acid., e.g., AA=4-Amb, 4-Apac, Lys, etc.
  MISC_FEATURE (9)..(9): R = H, Me etc.
  MISC_FEATURE (10)..(10): R = H, Me etc.
  MISC_FEATURE (10)..(10): Carboxy terminal group (-CO2H) is replaced with -CONR, where R is benzyl which is optionally substituted in 3′-, and 5′- position of the phenyl ring with H, CH3, CF3, etc. and the methylene group of the benzyl is optionally substituted with H, CH3, etc.
XPXXXXXPLW

SEQ ID NO: 44 - protein, 7 aa, Artificial Sequence
  Compound 44
  MISC_FEATURE (7)..(7): Carboxy group (-CO2H) is replaced with -CONHR, where R is 3′,5′-ditrifluoromethylbenzyl
YPWFPLW

SEQ ID NO: 45 - protein, 7 aa, Artificial Sequence
  Compound 45
  MISC_FEATURE (7)..(7): Carboxy group (-CO2H) is replaced with -CONHR, where R is 3′,5′-ditrifluoromethylbenzyl
YPFFPLW

SEQ ID NO: 46 - protein, 7 aa, Artificial Sequence
  Compound 46
  MISC_FEATURE (4)..(4): alpha-amino group is substituted with methyl
  MISC_FEATURE (7)..(7): Carboxy group (-CO2H) is replaced with -CONHR, where R is 3′,5′-ditrifluoromethylbenzyl
YPWFPLW

SEQ ID NO: 47 - protein, 7 aa, Artificial Sequence
  Compound 47
  MISC_FEATURE (4)..(4): alpha-amino group is substituted with methyl
  MISC_FEATURE (7)..(7): Carboxy group (-CO2H) is replaced with -CONHR, where R is 3′,5′-ditrifluoromethylbenzyl
YPFFPLW

SEQ ID NO: 48 - protein, 7 aa, Artificial Sequence
  Compound 48
  MISC_FEATURE (3)..(3): alpha-amino group is substituted with methyl
  MISC_FEATURE (4)..(4): alpha-amino group is substituted with methyl
  MISC_FEATURE (7)..(7): Carboxy group (-CO2H) is replaced with -CONHR, where R is 3′,5′-ditrifluoromethylbenzyl
YPWFPLW

SEQ ID NO: 49 - protein, 7 aa, Artificial Sequence
  Compound 49
  MISC_FEATURE (3)..(3): alpha-amino group is substituted with methyl
  MISC_FEATURE (4)..(4): alpha-amino group is substituted with methyl
  MISC_FEATURE (7)..(7): Carboxy group (-CO2H) is replaced with -CONHR, where R is 3′,5′-ditrifluoromethylbenzyl
YPFFPLW

SEQ ID NO: 50 - protein, 7 aa, Artificial Sequence
  Compound 50
  MISC_FEATURE (2)..(2): D-isomer
  MISC_FEATURE (7)..(7): Carboxy group (-CO2H) is replaced with -CONHR, where R is 3′,5′-ditrifluoromethylbenzyl
YAGFPLW

SEQ ID NO: 51 - protein, 6 aa, Artificial Sequence
  Compound 51
  MISC_FEATURE (2)..(2): D-isomer
  MISC_FEATURE (6)..(6): Carboxy group (-CO2H) is replaced with -CONHR, where R is 3′,5′-ditrifluoromethylbenzyl
YAFPLW

SEQ ID NO: 52 - protein, 7 aa, Artificial Sequence
  AKG190
  MISC_FEATURE (2)..(2): D-isomer
  MISC_FEATURE (4)..(4): 4-position of the phenyl group of the phenylalanine is substituted with fluoride
  MISC_FEATURE (7)..(7): Carboxy group (-CO2H) is replaced with -CONHR, where R is 3′,5′-ditrifluoromethylbenzyl
YAGFPLW

SEQ ID NO: 53 - protein, 7 aa, Artificial Sequence
  AKG180
  MISC_FEATURE (1)..(1): Dmt
  MISC_FEATURE (2)..(2): D-isomer
  MISC_FEATURE (3)..(3): alpha-amino group is substituted with methyl
  MISC_FEATURE (7)..(7): Carboxy group (-CO2H) is replaced with -CONHR, where R is 3′,5′-ditrifluoromethylbenzyl
XAGFPLW

SEQ ID NO: 54 - protein, 7 aa, Artificial Sequence
  AKG181
  MISC_FEATURE (1)..(1): Dmt
  MISC_FEATURE (2)..(2): D-isomer
  MISC_FEATURE (7)..(7): Carboxy group (-CO2H) is replaced with -CONHR, where R is 3′,5′-ditrifluoromethylbenzyl
XAGFPLW

SEQ ID NO: 55 - protein, 7 aa, Artificial Sequence
  AKG182
  MISC_FEATURE (1)..(1): Dmt
  MISC_FEATURE (2)..(2): D-isomer
  MISC_FEATURE (3)..(3): alpha-amino acid group is methylated
  MISC_FEATURE (4)..(4): alpha-amino acid group is methylated
  MISC_FEATURE (7)..(7): Carboxy group (-CO2H) is replaced with -CONHR, where R is 3′,5′-ditrifluoromethylbenzyl
XAGFPLW

SEQ ID NO: 56 - protein, 7 aa, Artificial Sequence
  AKG183
  MISC_FEATURE (1)..(1): Dmt
  MISC_FEATURE (2)..(2): D-isomer
  MISC_FEATURE (2)..(2): alpha-amino group is methlated
  MISC_FEATURE (3)..(3): alpha-amino group is methlated
  MISC_FEATURE (7)..(7): Carboxy group (-CO2H) is replaced with -CONHR, where R is 3′,5′-ditrifluoromethylbenzyl
XAGFPLW

SEQ ID NO: 57 - protein, 7 aa, Artificial Sequence
  AKG184
  MISC_FEATURE (1)..(1): Dmt
  MISC_FEATURE (2)..(2): D-isomer
  MISC_FEATURE (2)..(2): alpha-amino group is methylated
  MISC_FEATURE (4)..(4): alpha-amino group is methylated
  MISC_FEATURE (7)..(7): Carboxy group (-CO2H) is replaced with -CONHR, where R is 3′,5′-ditrifluoromethylbenzyl
XAGFPLW

SEQ ID NO: 58 - protein, 7 aa, Artificial Sequence
  AKG185
  MISC_FEATURE (1)..(1): Dmt
  MISC_FEATURE (2)..(2): D-isomer
  MISC_FEATURE (2)..(2): alpha-amino group is methylated
  MISC_FEATURE (3)..(3): alpha-amino group is methylated
  MISC_FEATURE (4)..(4): alpha-amino group is methylated
  MISC_FEATURE (7)..(7): Carboxy group (-CO2H) is replaced with -CONHR, where R is 3′,5′-ditrifluoromethylbenzyl
XAGFPLW

SEQ ID NO: 59 - protein, 8 aa, Artificial Sequence
  TY045
  MISC_FEATURE (2)..(2): D-isomer
  MISC_FEATURE (5)..(5): Nle
  MISC_FEATURE (8)..(8): Carboxy group (-CO2H) is replaced with -CONHR, where R is 3′,5′-ditrifluoromethylbenzyl
YAGFXPLT

SEQ ID NO: 60 - protein, 8 aa, Artificial Sequence
  AKG112
  MISC_FEATURE (2)..(2): D-isomer
  MISC_FEATURE (4)..(4): alpha-amino group is methylated
  MISC_FEATURE (5)..(5): Nle
  MISC_FEATURE (8)..(8): Carboxy group (-CO2H) is replaced with -CONHR, where R is 3′,5′-ditrifluoromethylbenzyl
YAGFXPLW

SEQ ID NO: 61 - protein, 8 aa, Artificial Sequence
  AKG113
  MISC_FEATURE (2)..(2): D-isomer
  MISC_FEATURE (4)..(4): alpha-amino group is methylated
  MISC_FEATURE (8)..(8): Carboxy group (-CO2H) is replaced with -CONHR, where R is 3′,5′-ditrifluoromethylbenzyl
YAGFGPLW

SEQ ID NO: 62 - protein, 8 aa, Artificial Sequence
  AKG130
  MISC_FEATURE (2)..(2): D-isomer
  MISC_FEATURE (4)..(4): 4-position of the phenyl group is substituted with fluoride
  MISC_FEATURE (8)..(8): Carboxy group (-CO2H) is replaced with -CONHR, where R is 3′,5′-ditrifluoromethylbenzyl
YAGFGPLW